(19) United States
(12) Patent Application Publication
(10) Pub. No.: US 2020/0222630 A1
(43) Pub. Date: Jul. 16, 2020

(54) METHODS AND DEVICES FOR FLUID DELIVERY AND ANALYTE SENSING VIA AN IMPLANTABLE PORT

(71) Applicant: TheraNova, LLC, San Francisco, CA (US)

(72) Inventors: Michael HEMATI, San Francisco, CA (US); Romain ROUX, Vancouver (CA); Daniel R. BURNETT, San Francisco, CA (US)

(73) Assignee: TheraNova, LLC, San Francisco, CA (US)

(21) Appl. No.: 16/821,480

(22) Filed: Mar. 17, 2020

Related U.S. Application Data
(63) Continuation of application No. PCT/US2018/051546, filed on Sep. 18, 2018.
(60) Provisional application No. 62/560,559, filed on Sep. 19, 2017.

(51) Int. Cl. A61M 5/172; A61B 5/00; A61B 5/145; A61M 5/142; A61M 39/02

(52) U.S. Cl. CPC A61M 5/1723; A61B 5/6865; A61B 5/6852; A61B 5/1459; A61B 5/14532; A61M 5/14248; A61M 39/0208; A61B 5/4839

(57) ABSTRACT

Methods and devices for fluid delivery and analyte sensing via an implantable port are disclosed where a port apparatus may be configured for placement within a body of a subject and may generally comprise a port housing and a catheter defining a first lumen which is fluidly coupled to the port housing. A flushing lumen may extend from the port housing and terminate at or near a distal tip of the catheter and an access port may be positioned within or upon the port housing and in fluid communication with the first lumen. Furthermore, an access device configured for percutaneous advancement into contact with the access port may also be included.

### CROSS-REFERENCE TO RELATED APPLICATIONS

This application is a continuation of International Application No. PCT/US2018/051546 filed Sep. 18, 2018, which claims the benefit of priority to U.S. Provisional Application No. 62/560,559 filed Sep. 19, 2017, the contents of which are incorporated herein by reference in their entireties.

### FIELD OF THE INVENTION

The present invention relates to the field of continuous biochemical monitoring and management systems. More specifically, the present invention relates to continuous glucose measuring devices and insulin infusion devices with minimal lag.

### INCORPORATION BY REFERENCE

All publications and patent applications mentioned in this specification are herein incorporated by reference to the same extent as if each such individual publication or patent application were specifically and individually indicated to be so incorporated by reference.

### BACKGROUND OF THE INVENTION

Diabetes is a group of diseases characterized by high levels of blood glucose resulting from defects in insulin production, insulin action, or both. Diabetes is the leading cause of blindness in people ages 20 to 70 and is sixth leading cause of death in the United States. Overall, the risk for death among people with diabetes is about 2 times that of people without diabetes. The disease often leads to other complications such as kidney, nerve and heart disease and strokes. It is the leading cause for non-traumatic amputations and kidney failure.

Diabetes is reaching epidemic proportions in the United States. There are approximately 18.2 million people in the United States, or 6.3% of the population, who have diabetes. While an estimated 13 million have been diagnosed with diabetes, 5.2 million people (or nearly one-third) are unaware that they have the disease. Furthermore, diabetes is one of the most common chronic diseases in children and adolescents; about 151,000 people below the age of 20 years have diabetes.

Diabetics must diligently monitor the glucose level in their blood. Blood glucose levels should be maintained between 80 to 120 mg/dl before meals and between 100-140 mg/dl at bedtime. Self-monitoring of blood glucose permits diabetics to know their blood sugar level so they can adjust their food, insulin, or activity level accordingly. Improved glucose control can forestall, reduce, or even reverse some of the long-term complications of diabetes.

The gold standard for testing blood glucose is the measurement of glucose in a plasma sample obtained from a vein. A drop of blood is placed on a small window in a test strip. Blood glucose acts as a reagent in a chemical reaction that produces a color change or generates electrons. The color change is detected by a reflectance-meter and reported as a glucose value. Alternatively, the electrons generated in the reaction are detected as an electrical current and reported as a glucose value.

Problems with these types of glucose measuring devices include the requirement of a drop of blood for each test (normally acquired through a prick of the finger). The blood sampling can be painful and cause calluses to form. It also increases the risk for warts and infections. The acute discomfort associated with this presents the largest barrier to life-saving blood glucose control.

Minimally invasive technologies currently include the GlucoWatch Biographer (no longer sold) and the Guardian® (registered trademark of Medtronic Minimed, Inc.) Continuous Glucose Monitoring System.

The GlucoWatch Biographer uses reverse iontophoresis, which involves applying an electrical microcurrent to the skin. The current pulls sodium through the intact skin, water follows sodium and water pulls glucose with it. The glucose concentration in this fluid is proportionate to the concentration in blood.

However, there are several problems with this technology. There is a lag time of 20 minutes before a blood glucose value can be reported. The concentration of glucose in the fluid is only 1/1,000 of glucose in the blood. A mild skin discomfort last for a few minutes when the device is first applied to the skin. The device is intended for use only by adults (age 18 and older) with diabetes. It is intended to supplement, not replace, standard home blood glucose monitoring devices. The user also has to calibrate the GlucoWatch Biographer with a blood glucose value measured on a traditional, i.e. “fingerstick,” monitor. Thus a standard (invasive) blood glucose monitor is still required.

The Guardian® Continuous Glucose Monitoring System is designed to automatically and frequently monitor glucose values in subcutaneous interstitial fluid (ISF). It measures ISF glucose every five minutes and it has a hypoglycemia alert. Once inserted, the sensor is virtually painless, but it requires entry of glucose readings from a standard monitor at least twice a day in order to calibrate the sensor. Furthermore, the readings from this monitor lag the actual blood glucose values by 15-20 minutes potentially resulting in over or under dosing of insulin.

Other marketed devices include a subcutaneously inserted continuous glucose monitor which functions for several days before requiring replacement. These devices, though, measure interstitial blood glucose which frequently lags blood glucose by 15 minutes or more.

This lag time is suboptimal (more manageable lag times are in the 5-10 minute range). More importantly, the lag times for glucose measurements using subcutaneous sensors is not consistent. As a result, no one control algorithm can be used to create a closed-loop system. The inter- and intra-sensor variability in lag time is too great (5-30 minutes according to some reports) and doesn't apply to each sensor the same way or even apply to the same sensor during certain physiological situations.

Subcutaneous glucose sensors are generally placed at least weekly in the subcutaneous space. A sensor placed one week may be placed near a capillary bed (lag time 5-10 min) while the sensor implanted a week later may be placed against a muscle fiber or fat tissue (30 minute or greater lag time). Therefore, the same control algorithm will not work adequately for both sensor placements. With respect to intra-sensor variability, many conditions affect blood flow to the submucosa of the skin. Cold temperature, for example, will drastically impact blood flow to the skin, and therefore have an effect on sensor readings. Sleeping also potentially impacts blood flow, and therefor subcutaneous sensor readings. Significant intra-sensor variability may exist between sleeping lag times and waking lag times. This variability may be due to episodes of severe nocturnal hypoglycemia.

### SUMMARY OF THE INVENTION

The intraperitoneal (IP) space has been shown to have more effective, faster insulin delivery and faster glucose sensing kinetics than the subcutaneous space. Various anatomical locations have been evaluated for blood glucose measurements, such as saliva and tears, and have been deemed inadequate for a closed, loop system due to latent lag times and interferences. The peritoneum, a thin transparent membrane that lines the walls of the abdominal cavity, contains the abdominal organs, and the fluids within the peritoneum are constantly exchanged by blood exudate. By comparison, subcutaneous tissues are located just below the skin surface and experience much lower blood perfusion rates. The IP space provides superior kinetics and a better medium for real-time glucose measurement. In some embodiments herein, insulin is delivered to the true pelvis.

Use of the peritoneal space provides a more direct tracking of blood glucose, capturing faster glucose kinetics, avoiding membrane/encapsulation effects, having less lag time and lag time variability, and eliminating the effect of variations in skin temperature, cardiac output, and body position during sleep. It would be implanted in an outpatient surgical center in a procedure similar to implantation of a peritoneal dialysis catheter. 2-fold faster glucose sensing kinetics can be achieved by placing a continuous glucose monitor in the intraperitoneal space versus the subcutaneous space. Sensing kinetics and sensor encapsulation are main factors contributing to accuracy and reliability of continuous blood glucose monitoring.

The peritoneal sensor system disclosed herein overcomes the inter- or intra-sensor lag time variabilities due to the consistent turnover of peritoneal fluid under most normal circumstances. Another potential advantage is the one-time placement of the device in a protected and/or fixed position within the peritoneal cavity. In addition a cleaning feature may be incorporated into the device to decrease the impact of fibrotic ingrowth and biofilm formation on the sensor. The present invention provides shorter lag times and better control of analyte/glucose measurements, and also provides a consistency that is critical to closed-loop control of blood glucose levels, especially when coupled with an insulin pump. Insulin may be delivered to the peritoneal space as well, or it may be delivered elsewhere, such as subcutaneously.

Potential spaces within the body experience a diminished immune response compared to that of the skin or subcutaneous spaces. Positioning a sensor within a potential space, for example, within the peritoneal cavity, rather than subcutaneously, reduces the immune response to the device. However, implanting a device more deeply within the body, and communicating with the device from outside the body can be challenging. The peritoneal sensor system disclosed herein overcomes these challenges and enjoys a reduced immune response, a longer implant life, and shorter lag times than current devices.

The peritoneal sensor system allows for the sensing or sampling component to be tunneled or place in a potential space with a catheter/tether connecting it to a controller/transmitter within the subcutaneous or pre-peritoneal space, or outside the body. This not only allows for data and power transmission from the controller to the sensor/sampling component, but also allows for easier retrieval and swapping of the sensor/sampling component with a minimally invasive procedure. Ideally the sensor/sampling component implant or swapping procedure can be performed over a guidewire. The sensor/sampling component may be detached from the controller and a replacement sensor/sampler may be reattached to the controller. In some embodiments, the sensor/sampler is accompanied by an insulin infusion catheter or lumen to provide not only the faster and more durable sensing benefit of the peritoneal space, but also the faster insulin absorption benefit of the peritoneal space.

Generally, the peritoneal sensor system may comprise a catheter having a distal tip which is tapered, and a sensor positioned within the tapered distal tip of the catheter, wherein the sensor is configured to sense for a presence of one or more analytes when positioned within peritoneal fluid of a subject. A filter may be in fluid communication with the sensor may also be included, wherein the filter is permeable to the one or more analytes, as well as a controller in communication with the sensor and a port in fluid communication with the catheter and the sensor, wherein infusion of a fluid through the port flushes the sensor with the fluid.

In yet another embodiment, the peritoneal sensor system may comprise a catheter having a distal tip which is tapered, and the sensor configured to sense for a presence of one or more analytes when contacting peritoneal fluid of a subject. A controller in communication with the sensor may also be included, wherein the sensor is positioned within the controller, as well as a filter in fluid communication with the sensor, wherein the filter is permeable to the one or more analytes and a port in fluid communication with the catheter and the sensor, wherein infusion of a fluid through the port flushes the filter with the fluid.

In use, the sensor system may be used for detecting one or more analytes within a subject, generally comprising contacting peritoneal fluid within the subject via a distal tip of a catheter, where the distal tip is tapered to inhibit or reduce an ability of a fibrotic capsule from obtaining purchase, filtering the peritoneal fluid, wherein the filter is permeable to the one or more analytes, sensing for a presence of one or more analytes within the peritoneal fluid via a sensor, determining whether the one or more analytes are present within the peritoneal fluid via a controller in communication with the sensor, and infusing a fluid within the catheter such the fluid flushes the distal tip.

Any of the embodiments detailed herein can be used in any potential space, including, but not limited to, the pleural space, the cerebral spinal fluid space, the peritoneal space, the true pelvis, etc.

Any of the embodiments detailed herein may include a sensor in the potential space, and/or a sampler in the potential space. In some embodiments, a fluid/analyte sampler is in the potential space, and a sensor is in the controller which may be in the subcutaneous or pre-peritoneal space or external to the patient.

In one embodiment, the port apparatus may be configured for placement within a body of a subject and may generally comprise a port housing and a catheter defining a first lumen which is fluidly coupled to the port housing. A flushing lumen may extend from the port housing and terminate at or near a distal tip of the catheter and an access port may be positioned within or upon the port housing and in fluid communication with the first lumen. Furthermore, an access device configured for percutaneous advancement into contact with the access port may also be included.

In another aspect, the port apparatus may be configured for subcutaneous placement within the body.

In another aspect, the port apparatus may further comprise a reservoir configured to be placed into fluid communication within the access port. In another aspect, the reservoir may comprise a syringe. In another aspect, the reservoir contains insulin.

In another aspect, the catheter may comprise a distal tip which is configured for positioning within a peritoneal space of the body. In another aspect, the distal tip may be configured for positioning within a true pelvis of the peritoneal space.

In another aspect, the port apparatus may further comprise an analyte sensor positioned to be in communication with a fluid within the body. In another aspect, the analyte sensor may be positioned within the port housing. In another aspect, the analyte sensor may be positioned within or upon the catheter.

In another aspect, the port apparatus may further comprise a controller in communication with the port apparatus. In another aspect, an analyte sensor may be positioned within the controller.

In another aspect, the controller may be configured to control an infusion of insulin through the catheter and into the body and/or monitor a glucose level of the body.

In another aspect, the controller may be configured to infuse a fluid through the catheter in a closed loop or semi-closed loop based upon a parameter sensed within a fluid contained within the body.

In another aspect, the controller may be configured to monitor a parameter of a peritoneal fluid drawn from within a peritoneal cavity of the body.

In another aspect, the analyte sensor may be in communication with the controller for detecting at least one parameter within a fluid received through the lumen.

In another aspect, the controller may be configured for maintaining the access device in place within the access port for at least 1 hour.

In another aspect, the controller may be configured for maintaining the access device in place within the access port continuously.

In another aspect, the controller may be configured to periodically provide a supply of insulin and to continuously detect a glucose level of the body via the access device.

In another aspect, the analyte sensor may comprise a glucose sensor.

In another aspect, the analyte sensor may be incorporated into the catheter.

In another aspect, the analyte sensor may be incorporated into the port housing.

In another aspect, the analyte sensor may be positioned remotely from the port apparatus.

In another aspect, the access device may comprise at least one conductive portion configured to be in electrical communication with the access port.

In another aspect, the access device may comprise two or more conductive portions. In another aspect, the two or more conductive portions may be configured to be in electrical communication with two or more conductive portions of the access port in a corresponding manner.

In another aspect, the access device may further comprise a patch configured for securement to a skin surface of the subject.

In another aspect, the access device may comprise a needle or cannula having at least one conductive portion configured to be in electrical communication with the access port.

In another aspect, the port apparatus may further comprise a flushing mechanism in fluid communication with the catheter, wherein the flushing mechanism is configured to apply a negative and/or positive pressure via the catheter.

In another embodiment, the port apparatus may generally comprise a port housing configured for subcutaneous placement within a body of a subject and a catheter defining a lumen which is fluidly coupled to the port housing. A flushing lumen may extend from the port housing and terminate at or near a distal tip of the catheter and an access port may be positioned within or upon the port housing and in fluid communication with the lumen. An analyte sensor may also be in electrical communication with the access port and an access device configured for percutaneous advancement into contact with the access port may also be included such that the access device and access port are in electrical communication when the access device is received within the access port. Additionally, a controller may be in electrical communication with the access device such that the controller is in communication with the analyte sensor when the access device is in electrical communication with the access port.

In another aspect, the access device may comprise at least one conductive portion configured to be in electrical communication with the access port.

In another aspect, the access port may comprise at least one conductive portion configured to contact the at least conductive portion of the access device.

In another aspect, the access device may comprise two or more conductive portions. In another aspect, the two or more conductive portions may be configured to be in electrical communication with two or more conductive portions of the access port in a corresponding manner.

In another aspect, the port apparatus may further comprise a reservoir configured to be placed into fluid communication within the access port. In another aspect, the reservoir may comprise a syringe. In another aspect, the reservoir may contain insulin.

In another aspect, the catheter may comprise a distal tip which is configured for positioning within a peritoneal space of the body.

In another aspect, the distal tip may be configured for positioning within a true pelvis of the peritoneal space.

In another aspect, the port apparatus may further comprise a flushing mechanism in fluid communication with the catheter, wherein the flushing mechanism is configured to apply a negative and/or positive pressure via the catheter.

In another aspect, the analyte sensor may comprise a glucose sensor.

In another aspect, the analyte sensor may be incorporated into the catheter.

In another aspect, the analyte sensor may be incorporated into the port housing.

In another aspect, the controller may be configured to control an infusion of insulin through the catheter and into the body and/or monitor a glucose level of the body.

In another aspect, the controller may be configured to infuse a fluid through the catheter in a closed loop or semi-closed loop based upon a parameter sensed within a fluid contained within the body.

In another aspect, the controller may be configured to monitor a parameter of a peritoneal fluid drawn from within a peritoneal cavity of the body.

In another aspect, the port apparatus may further comprise an analyte sensor in communication with the controller for detecting at least one parameter within a fluid in contact with the analyte sensor.

In another aspect, the access port may comprise a plurality of conductive mesh sheets. In another aspect, the plurality of conductive mesh sheets may be configured to be aligned in a corresponding manner with the access device.

In one example for a method of controlling an infusion of fluid within a body of a subject may generally comprise receiving an access device advanced percutaneously into contact with an access port positioned within or upon a port housing, wherein the port housing is positioned subcutaneously within a body of a subject, forming an electrical contact between at least one conductive portion of the access device and at least one conductive portion of the access port, and contacting a fluid from within the body with an analyte sensor. The method may also include receiving a signal from the analyte sensor in contact with the fluid and determining at least one parameter from the fluid relating to the body via a controller in electrical communication with the analyte sensor.

In another aspect, the access device may comprise at least one conductive portion configured to be in electrical communication with the access port.

In another aspect, the method may further comprise drawing the fluid from within a peritoneal cavity into a catheter fluidly coupled to the access port.

In another aspect, the access port may comprise at least one conductive portion configured to contact the at least conductive portion of the access device.

In another aspect, forming an electrical contact may comprise electrically contacting two or more conductive portions along the access device with two or more conductive portions of the access port in a corresponding manner.

In another aspect, the method may further comprise introducing a volume of insulin through the catheter and into the body.

In another aspect, the volume of insulin may be based upon the at least one parameter.

In another aspect, the catheter may comprise a distal tip which is configured for positioning within a peritoneal space of the body. In another aspect, the distal tip may be configured for positioning within a true pelvis of the peritoneal space.

In another aspect, the method may further comprise flushing a second fluid into the catheter.

In another aspect, the determining at least one parameter may comprise determining a glucose level within the body.

### BRIEF DESCRIPTION OF THE DRAWINGS

FIG. 1 shows an embodiment of the peritoneal sensor system with a catheter/tether which includes wireless communication.

FIG. 2 shows another embodiment of the peritoneal sensor system.

FIG. 3 shows an embodiment of the peritoneal sensor system with an externalized control portion.

FIGS. 4A-4C show some example embodiments that incorporate flushing.

FIG. 5 shows the relative responsiveness of sensors implanted in the peritoneal cavity, subcutaneous space and in the intravenous space.

FIG. 6A shows another embodiment of a peritoneal sensor system.

FIG. 7 shows an embodiment of the peritoneal sensing system with an anti-adhesion cuff.

FIGS. 8A-8D show embodiments which include mechanisms to clear fiber/fibrin and encapsulation.

FIGS. 9A-9D shows an embodiment which includes an expandable mesh-like, or cage-like, component.

FIGS. 10A-10D show embodiments where the sensor assembly component includes an outer collar, or sleeve, over the sensor catheter assembly.

FIG. 11 shows an embodiment with a separately implantable “landing pad”, or base structure.

FIG. 12 shows an embodiment which includes a shroud, or cover, over the sensor.

FIG. 13 shows an embodiment with multiple sensors.

FIG. 14 shows an embodiment with an array of sensors.

FIG. 15 shows an embodiment of the peritoneal sensor system which uses spectroscopy or spectrophotometry.

FIG. 16 shows another embodiment which uses spectroscopy or spectrophotometry.

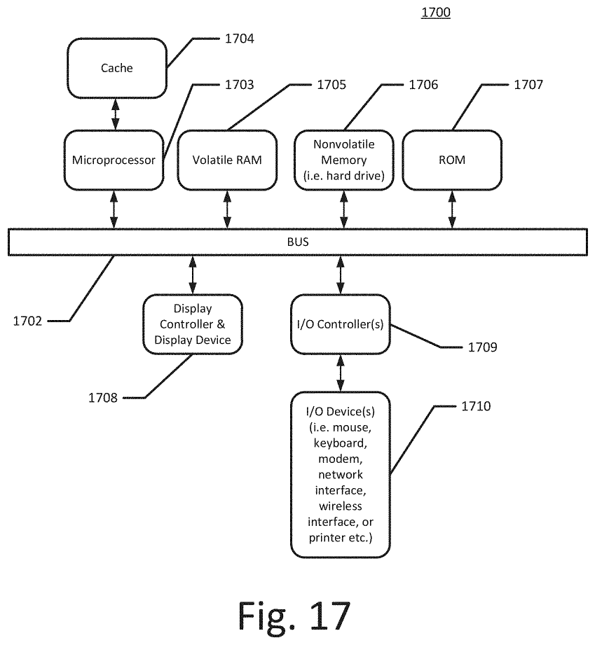

FIG. 17 shows a block diagram of a data processing system, which may be used with any embodiments of the invention.

FIG. 18 shows an embodiment of a peritoneal insulin delivery system.

FIG. 19 shows detail of a subcutaneous port.

FIG. 20 shows an embodiment of a subcutaneous port with a large reservoir.

FIGS. 21A and 21B show embodiments of a subcutaneous port with a deep reservoir.

FIG. 22 shows an embodiment of a subcutaneous port with a septum with a larger surface area.

FIGS. 23 and 24 show an embodiment of the insulin delivery system where the needle is removed after the patch has been placed on/in the skin.

FIG. 25 shows an embodiment which combines insulin delivery and peritoneal cavity sensing.

FIGS. 26A and B show embodiments of the insulin delivery system which may be particularly useful with overweight patients.

FIGS. 27A-C show embodiments of the insulin delivery system which may incorporate flushing of the system, as well as analyte sensing of fluid within the peritoneal cavity.

FIGS. 27D-E show embodiments of the insulin delivery system which incorporate a separate flushing reservoir and lumen.

FIGS. 27F-I show embodiments of the insulin delivery system which incorporate a smart needle and/or cannula.

FIGS. 28A-E show various embodiments of the insulin delivery system which include features to help maintain the needle 814 within the reservoir of the subcutaneous port.

FIGS. 29A-C show embodiments that incorporate a mesh in the subcutaneous port.

FIGS. 30A-F show alternative needle capture embodiments of the insulin delivery system.

FIGS. 31A-B show an embodiments incorporating a balloon/expandable member.

FIGS. 31C and 31D show an embodiment that includes a shape memory needle.

FIGS. 31E-G show an embodiment utilizing a locking needle.

FIGS. 32A and B show an embodiment of a piercable septum.

FIG. 33 shows an embodiment of the port which includes a foam insert.

### DETAILED DESCRIPTION OF THE INVENTION

The peritoneal sensor system generally includes a sensor/sampler portion, which is implanted in the peritoneal space, and a control portion/controller, which may be implanted elsewhere, such as subcutaneously, or may be external to the patient. Other functions which may be included include insulin delivery, sensor flushing, wireless communication, light spectroscopy, UV sterilization, analyte sampling, analyte circulation, logic, etc.

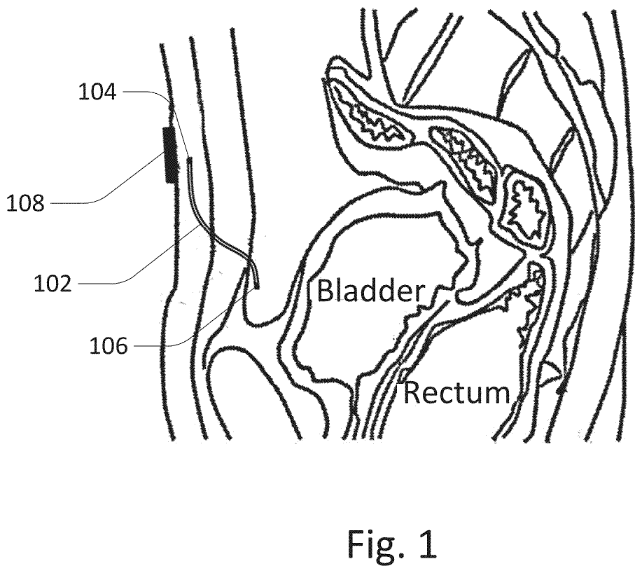

FIG. 1 shows an embodiment of the peritoneal sensor system with a tether/catheter which includes wireless communication. The embodiment of the peritoneal sensor system shown here includes catheter/tether 102 including subcutaneous antenna 104 on one end, and sensor portion 106 on the other end. For example, the sensor may sense the presence of glucose. This allows the sensor portion to communicate with external transmitting controller 108 where the sensor portion has low power requirements despite its position deep within the body. This design may incorporate an anchoring cuff or tunnel, for example, a cuff made of Dacron, to allow for tissue ingrowth and subsequent anchoring of catheter/tether to the body to hold it in place. The anchoring cuff may be anchored to a thin biocompatible tube or tunnel through which the sensor/catheter/tether assembly may be inserted from the subcutaneous space into the peritoneal space.

When the sensor has reached the end of its useful life, the antenna end of the catheter/tether may be accessed under local anesthesia, the sensor/catheter/tether assembly may be removed from the biocompatible tube through gentle traction and another sensor slid into the tube to replace the expired sensor. This assures consistent and easy placement of the sensor portion within the peritoneal cavity. Placement may also be accomplished by threading a guidewire down the center of the sensor/catheter/tether assembly, removing the sensor/catheter/tether assembly, then threading a new sensor/catheter/tether assembly over the guidewire. In this last embodiment, the tube or tunnel and/or anchoring cuff may not be necessary due to the formation of a fibrotic tunnel for the replacement sensor/catheter/tether assembly to follow.

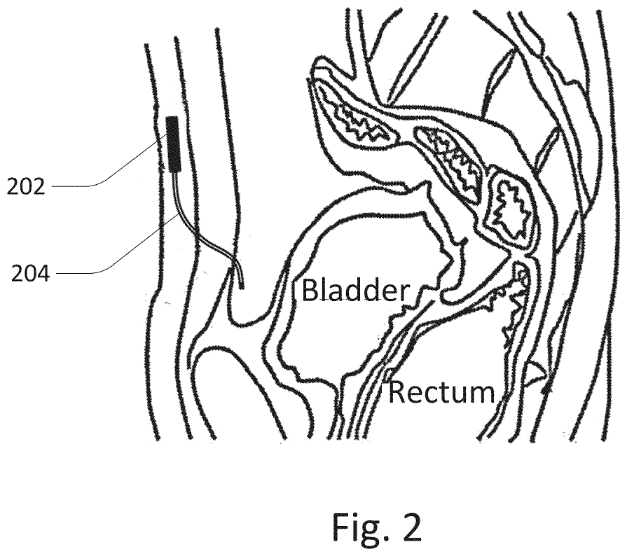

FIG. 2 shows another embodiment of the peritoneal sensor system. The system detailed in this Figure includes implanted controller portion 202, shown here in the subcutaneous space. The controller portion may alternatively be in the peritoneal or pre-peritoneal space. In this embodiment, sensor/catheter/tether assembly 204 is connected to implanted controller portion 202. In this embodiment, the control portion in the subcutaneous space may provide automated intermittent flushing of the sensor portion and provides power to the sensor portion and may store/transmit any collected data. Alternatively, flushing may be achieved manually or automatically via an implanted port using a syringe or other infusion device. The sensor portion may also or alternatively be flushed/refilled by an external port. Any of the embodiments may also allow for automatic or manual infusion of insulin based on the glucose readings in the peritoneal space. Insulin may be infused/refilled similarly to the flushing solution. Insulin and the flushing solution may be combined. The sensor/catheter/tether assembly may include a sensor, or it may simply sample fluid, causing it to flow to or past a sensor within the controller to determine the analyte concentration.

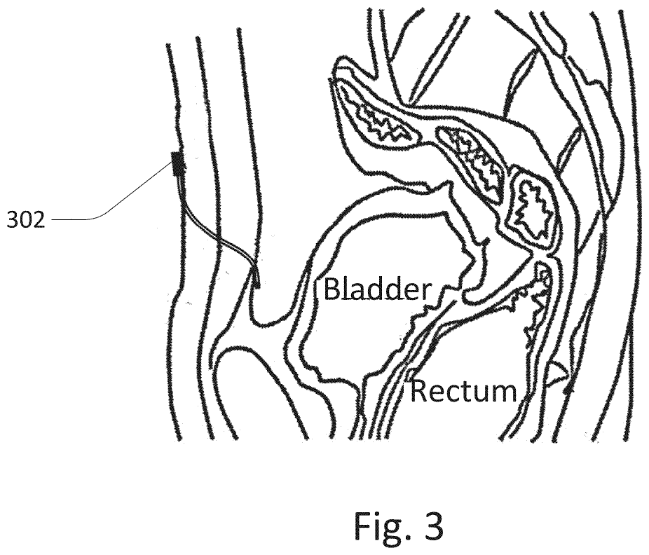

FIG. 3 shows the use of an externalized control portion. In this embodiment, control portion 302 may be externalized and interface with the sensor/catheter/tether portion outside of the body. The system may also incorporate sensor flushing and/or insulin infusion and may incorporate one or more anchor cuffs to prevent infection tracking. In this embodiment, the external control portion of the system may be very small and include a small chip and a small battery to collect, store and transmit the signals of the sensor portion of the system. The external control portion may be less than 5cc in volume and be relatively inconspicuous. The external control portion may also incorporate a small pump and a small insulin reservoir to provide a true artificial pancreas solution in a very low-profile design. All or part of the external control portion may be replaced and/or refilled on a daily, weekly or monthly basis to allow for a smaller, more compact profile.

The sensor portion may be replaced on a similar timeframe, or less frequently, to allow for a more stable signal over time. If the control portion (externalized or implanted) includes the ability to infuse insulin, the insulin may be infused at a site along the length of the tether/catheter that is in the peritoneal cavity, but is far enough away from the sensor to prevent signal disruption. For example, the insulin delivery exit may be about 0.5 cm to 1.5 cm from the sensor. Alternatively, the insulin may, itself, act as the flush for the sensor.

In any of the embodiments disclosed herein, any suitable sensing technology may be used, but ideally a sensor will be used that is both durable and resistant to micromotion and macromotion artifact. The sensor portion may include glycoenzymatic sensors with a membrane to prevent acute disruption of their surroundings. In some embodiments, the sensor portion includes a sensing modality that does not consume glucose such as infrared, raman spectroscopy, spectro-photometry, fluorescence (conA or boronate chemistry) or phosphorescence.

The terms, sensor, or sensing element, as disclosed herein, may also include a local or remote interface to a sensor. For example, a filter membrane which is permeable to glucose, but impermeable to other contaminates, may be used to filter fluid from the peritoneal cavity. The sensor in these embodiments may be in proximity to the filter membrane, or it may be remote to the filter membrane. For example, the filter membrane may be at the tip of the catheter assembly, but the sensor may be either at the proximal end of the catheter assembly, or in the control portion of the system. Alternatively the filter membrane may be in the controller or at the proximal end of the catheter. In these embodiments, a fluid column, or fluid reservoir, is in fluid communication with both the filter membrane, and the sensor.

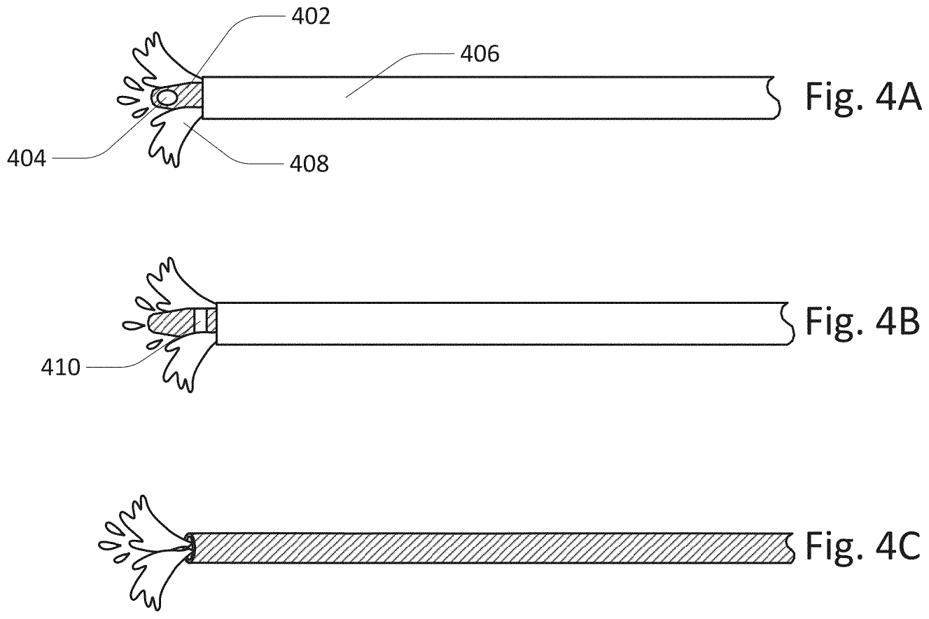

In some embodiments, the sensor/catheter/tether assembly may incorporate an insulin infusion lumen and/or a flushing lumen to keep the sensor free of encapsulation. The flushing lumen may be intermittently flushed from an internal reservoir of fluid, fluid from the peritoneal cavity, or fluid from an external source. Flushing may be performed automatically or manually. FIGS. 4A-4C show some example embodiments that incorporate flushing. The distal end of the catheter/sensor assembly is designed to prevent a fibrotic capsule from obtaining purchase and may therefore be either consistent in diameter or decreasing in diameter (moving proximally, to distally) and smooth, i.e. free of any trapped spaces, or indentations, to prevent trapping of fibrin and to allow a flush to readily remove any capsule forms. In other words, the tip of the catheter may be tapered so it is smaller toward the distal tip. The tip of the catheter is preferably made from a material which inhibits cell ingrowth such as silicone or other suitable material. This tapered tip feature may be incorporated into any of the embodiments disclosed herein.

FIG. 4A shows distal end 402 of the catheter/sensor assembly. The catheter assembly includes sensor or sensor interface 404. Flushing fluid, such as saline, peritoneal fluid, insulin or other fluid 408 is flushed through the annular lumen between flushing sleeve 406 and the catheter assembly. Note that in this embodiment the distal tip of the catheter assembly is smooth, and decreasing in diameter (moving proximally to distally). This helps the forces supplied by the flushing action to remove any fibrin capsule. Although the flushing lumen shown here is annular, the flushing lumen may also be round or any other shape, and may be along one or more than one side of the catheter assembly. Insulin may be infused through the flushing lumen, or through a separate lumen. The distal opening of the insulin lumen may be proximal to, and spaced apart from, the sensor at the distal end of the catheter assembly. A separate insulin lumen is not shown here.

FIG. 4B shows another embodiment of the peritoneal sensor system catheter assembly. In this embodiment, filter membrane 410 is shown. In this embodiment, filter membrane 410 may encircle the distal tip of the catheter assembly for maximum surface area exposure.

FIG. 4C shows an embodiment of the catheter assembly where the sensor is inside the lumen of the catheter assembly. In this embodiment, flushing may be performed through the same lumen as the lumen where the sensor resides.

In some embodiments of a flushing mechanism, the flushing solution may be used to flush the sensor portion to clear off encapsulation. In some embodiments of the sensor assembly described herein, the flushing fluid exits the flushing sleeve proximal to the sensing element and flushes the sensing element toward the tip, or distal end. Alternatively, the flushing solution may exit the tubing distal to the sensing element and flush the sensing element in the opposite direction, or proximally. Ensuring adequate fluid flow over the sensing element helps keep the sensing element clear and readings accurate and with minimal lag time. Flushing may occur continuously or intermittently.

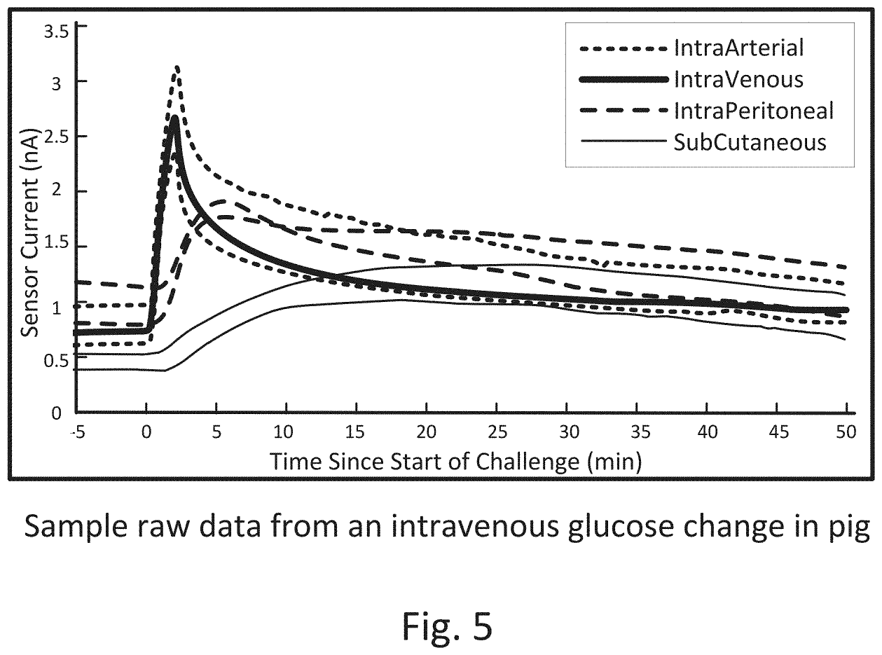

FIG. 5 shows the relative responsiveness of sensors implanted in the peritoneal cavity, subcutaneous space and in the intravenous space. The curves are shown following an Intravenous Glucose Tolerance Test (IVGTT). The sensor implanted in the peritoneal cavity resulted in a faster signal response than those implanted in the subcutaneous space. The response time of the sensor in the peritoneal cavity approached that of the intravenous sensor.

Micromotion and Noise Prevention:

Dynamic changes in the environment immediately surrounding the sensor may cause sensor signal noise. These dynamic changes may be a result of the peristaltic motion moving the sensor and/or micro motion associated with the sensors. These motion artifacts may adversely affect the signal of enzyme based sensors which rely on stable gradients of glucose, oxygen and H₂O₂ in order to generate a nanoampere current which can be translated into a blood glucose reading. Shifts in this local environment may cause significant changes in the nanoampere current which may adversely affect the resulting blood glucose signal. The peritoneal sensor system may reduce signal noise in some embodiments by controlling the environment near the sensor, using multiple sensors, and/or using a sensor which does not consume glucose.

Controlling the Environment Near the Sensor

A semi permeable membrane, or filter membrane, or other material may be used to filter the fluid which comes in contact with the sensor. This allows fluid to flow in the area local to the sensor and allows analytes (such as glucose) to diffuse in and out of the membrane, while preventing contaminates from doing so. In other words, the semi permeable membrane has pores that allow glucose (or any specific analyte) to pass through the pores, but larger items in the fluid cannot pass through the membrane. Alternatively, the membrane may allow only specific items to pass through it based on other characteristics other than size, for example electric charge, shape, etc. This technique increases the stability of the sensor signal, and also reduces contaminating components of the analyte fluid. Using a thin membrane, that encourages local diffusion in and out of the membrane over the order of seconds to minutes, allows the signal to be sufficiently stabilized to allow for the acquisition of the required data with acceptable lag times. The membrane may be near the sensor, for example covering the sensor, or the membrane may be remote to the sensor, with fluid communication between the membrane and the sensor.

Filter membranes may be made out of any suitable material known in the art, including the materials disclosed in U.S. Pat. Nos. 8,543,184, 7,613,491, and 8,050,731, each of which is hereby incorporated by reference in its entirety. The filter membrane may be hydrophilic or hydrophobic.

Multiple Sensors

In another embodiment, a “web” or array of sensors may be dispersed within the peritoneal cavity, for example around the pancreas, to collect glucose concentration data from multiple locations within the peritoneal cavity. In this embodiment data is collected from multiple locations. These values obtained from the multiple sensors may be averaged and signal noise is reduced.

Sensors which Don't Consume Glucose

Non-glucose consuming sensors are highly resistant to motion (both micromotion and the larger peristalsis-related motion) because they do not rely on detection of H₂O₂ in the local milieu of the sensor to maintain stability of readings. This type of sensor may include spectro-photometric, infrared, LED, raman spectroscopic, fluorescent or phosphorescent sensors or may rely on any other mechanism to detect glucose in a sample that does not consume the glucose or generate readings based on byproducts of an enzymatic reaction. The use of a non-enzymatic, non-glucose-consuming sensor in the peritoneal cavity generates stable readings with minimal signal lag times.

Encapsulation:

Implanted sensors will become encapsulated over time once implanted and the signal lag time will lengthen if the sensor is not flushed or otherwise cleaned. This increasing lag time can be found in sensors in either the peritoneal and subcutaneous spaces, but is more extreme in the subcutaneous space. Encapsulation has also been found to be more rapid and more extreme in the upper quadrant of the peritoneal cavity than in the lower quadrants (away from the omentum). Because of this, placing the sensor component in the pelvis (away from the omentum and liver) may be optimal. Alternatively, in the event that a patient has pelvic omentum, a method of catheter/sensor placement may be utilized which includes a procedure to tack the omentum up near the liver to keep the omentum away from the pelvic region.

In addition, we have discovered an unexpected finding during our intravenous glucose tolerance tests that indicate that during hyperglycemia (blood glucose >200 mg/dL) abnormally high glucose values were reported only in sensors in the upper quadrant of the peritoneal cavity. These values were in excess of the glucose values reported by capillary and plasma glucose. Conversely, the glucose readings of the pelvic peritoneal sensors tracked the readings of the capillary and plasma glucose readings more closely. Based on these data, it is possible that the liver “weeps” glucose into the peritoneal cavity when it is overwhelmed with hyperglycemia thereby creating falsely elevated glucose readings in sensors in that area. This could lead to excessive insulin administration which could be fatal. For this reason the sensor portion may not be placed in the traditional site of peritoneal cavity access for insulin infusion—the hepatic region.

In any system that senses glucose and delivers insulin, the glucose sensing may be more successfully accomplished at a distance away from the site of insulin delivery (which has traditionally been in the hepatic region). This may be accomplished with sensing in the pelvis and delivery of insulin in the hepatic region (a single dual lumen catheter or two single lumen catheters) or, alternatively, insulin delivery and glucose sensing both performed in the pelvis.

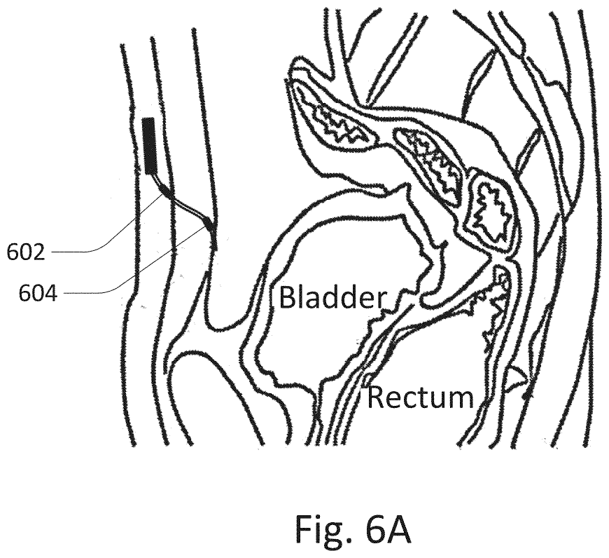

In some embodiments of the peritoneal sensor system, the peritoneal catheter (or catheter in any other potential space), may lie along the wall of the cavity and not protrude significantly into the space. This may prevent issues with catheter kinking, catheter movement due to peristalsis or direct force from the organs, and catheter obstruction/erosion due to direct organ contact. The catheter portion of the present invention may be placed in the pelvis with a short section of the catheter being tunneled through the rectus sheath or preperitoneal space prior to entry into the peritoneal cavity (see FIG. 6A). This allows for the catheter to be angled into the pelvis and away from the omentum to better ensure its continued patency and function. This placement can be used with either the analyte (such as glucose) sensor portion (or sampling portion) and/or drug (such as insulin) infusion portion of the system. These may be included on the same catheter or be separate catheters. Optional anchoring, or ingrowth, cuffs, 602 and 604, are shown in FIG. 6A.

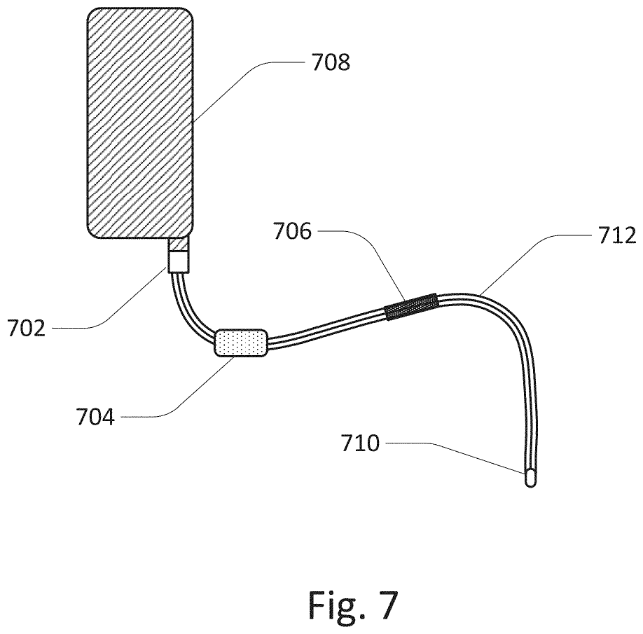

FIG. 7 shows an embodiment of the peritoneal sensing system with an anti-adhesion cuff. While all foreign bodies generate a foreign body response, different materials do so at different rates. For example, ePTFE of the appropriate pore size and silicone from a polished mold (or coated with PEG, albumin or other hydrophilic coatings) will be highly anti-adhesive. A short segment of anti-adhesion material, or an anti-adhesion cuff, shown as 706, on sensing catheter 712 will create a weakness in any fibrin capsule overlying this site. This weakness will allow the capsule to break at this point and be flushed from the distal end of the catheter with a fluid infusion or flush. The diameter of the distal end of the catheter is preferably constant in size or decreasing in size, and smooth, so that the catheter has no ridges or crenellations that will allow a capsule to invade and take hold. Drugs or other additives may also be added to the anti-adhesive cuff.

Also shown in FIG. 7 is reversible connector 702, one or more ingrowth cuffs 704 to prevent tracking of any infection or fluid along the catheter and controller 708. Tip 710 of infusion/sensing/sampling catheter 712 will ideally be blunt, and possibly weighted. Catheter 712 may include an insulin infusion sheath. Catheter 712 may include sterilization elements including electric current, silver, an ultraviolet light source etc. A glucose sensor may travel alongside, or within an insulin infusion sheath. Fluid, such as a dialysate, may be circulated within a dual lumen catheter, or forced in and out of a single lumen catheter, and drawn over a sensor via a pump. The sensor may be at the tip of the catheter, or it may be anywhere along the catheter, or it may be within the controller. Peritoneal fluid may also be drawn into the catheter to pass over the sensor.

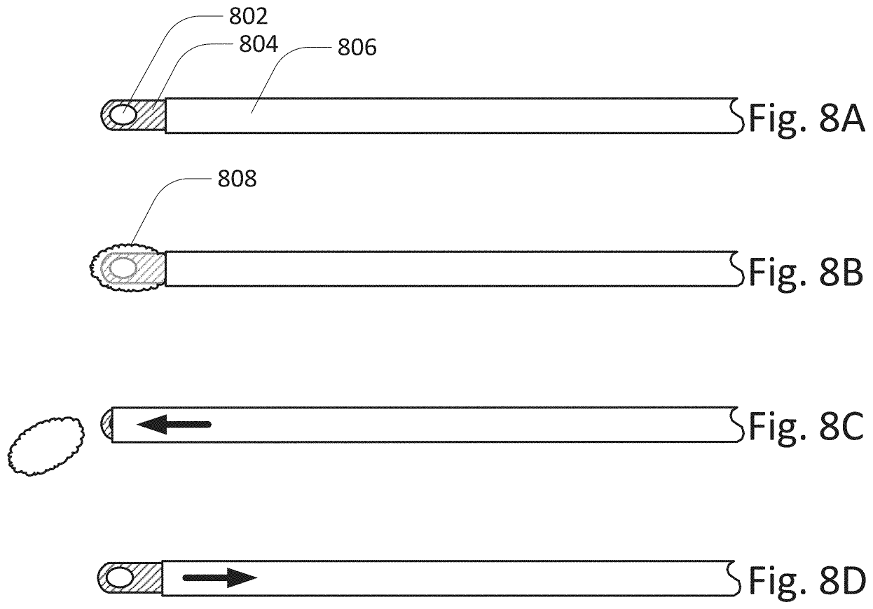

Other embodiments include additional mechanisms to clear fiber/fibrin and encapsulation. In one embodiment, the sensor body is assembled with an outer collar, or sleeve, that can be slid over the catheter body periodically to act as a wiper to physically remove any encapsulation growth 808, so that sensor 802 can function properly. See FIGS. 8A-8D. The collar/sleeve may run the length of sensor catheter 804, as shown here, or may be shorter than the sensing catheter, and controllable by moving catheter 804 relative to sleeve/cuff 806. The clearing action can be initiated by pulling the sensor assembly back while the collar stays in place or pushing the collar/sleeve forward while the sensor stays in place. This could be performed manually by the patient or via a hand pump. It could also be performed automatically using an air pump or motor actuation to push or pull the collar or sensor assembly.

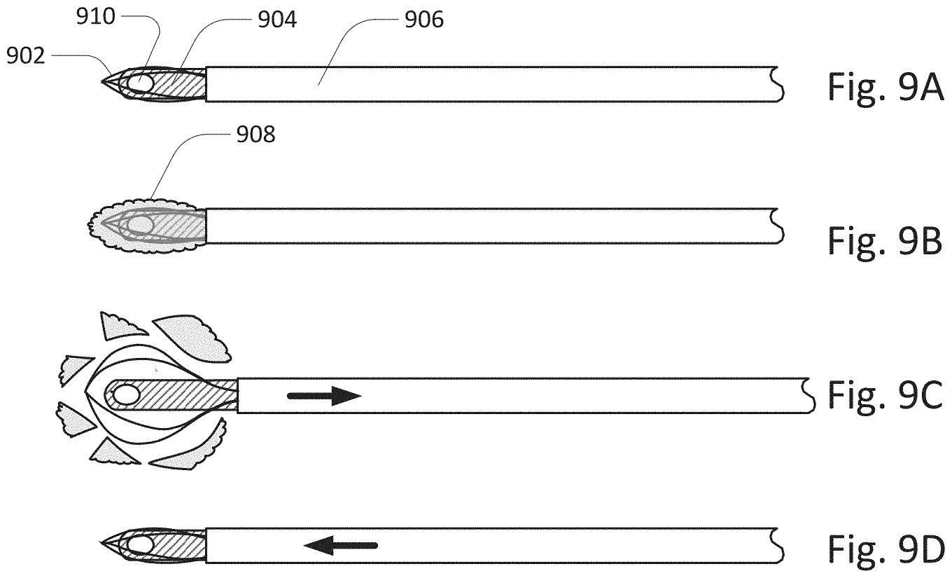

FIG. 9 shows an embodiment which includes an expandable mesh-like, or cage-like, component comprised of thin gauge, wire (e.g. stainless steel or Nitinol). Expandable cage component 902 has a retracted, or compressed, state, and an expanded state. FIGS. 9A and 9D shows the expandable cage in the compressed state. Because the cage is porous, webbed, mesh-like or has openings, sensor 910 can function normally with the cage in the compressed state. Fibrin 908 may form over distal end 904 of the catheter as shown in FIG. 9B. The expandable cage component may be held in the compressed state by sleeve 906. When sleeve 906 is moved proximally with respect to the cage, the cage is allowed to expand to its natural expanded state as shown in FIG. 9C. Alternatively, the cage may be moved distally with respect to sleeve 906 to expand the cage. The opposite move is performed to collapse the cage.

Alternatively, cage 902 may be connected at its distal end to the distal end of the sensing catheter and at its proximal end to sleeve 906. In this embodiment, sleeve 906 is moved distally with respect to the sensing catheter (or the catheter is moved proximally with respect to the sleeve) to expand the cage. The opposite move is performed to collapse the cage.

The cage may be similar to a device called a stone retrieval basket used to retrieve kidney stones. The expansion of the cage may be performed manually or automatically. Alternatively, a canvas like material or a balloon may be used instead of the cage.

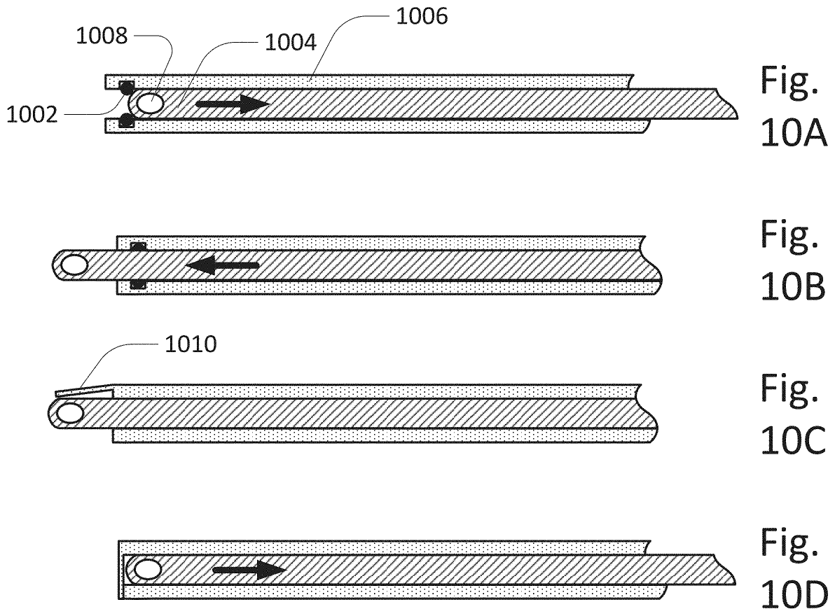

In the embodiments shown in FIGS. 10A-10D, the sensor assembly component may include an outer collar, or sleeve, over the sensor catheter assembly. Sleeve 1006 shields sensor 1008 and distal end 1004 of the catheter from encapsulation. The sensor may be extended outside of the sleeve when a measurement is required (e.g. once a minute), a measurement is taken, and the distal end of the sensor catheter assembly is retraced back within the sleeve. The sleeve incorporate a silicone membrane with a small hole, i.e. a sphincter, which expands as the sensor exits the collar. Alternatively, an O-ring may be used. FIGS. 10A and 10B show O-ring 1002. The sleeve may have a “trap door” that prevents fluid ingress during the time that the sensor is not active, and open to allow the sensor to exit the shroud. This opening could utilize spring force to open and close (spring, living hinge, etc.) and seal against the collar with an O-ring. Preferably, “trap door” 1010 is biased so that it is normally in the closed position, requiring a small amount of force to force it into the open position.

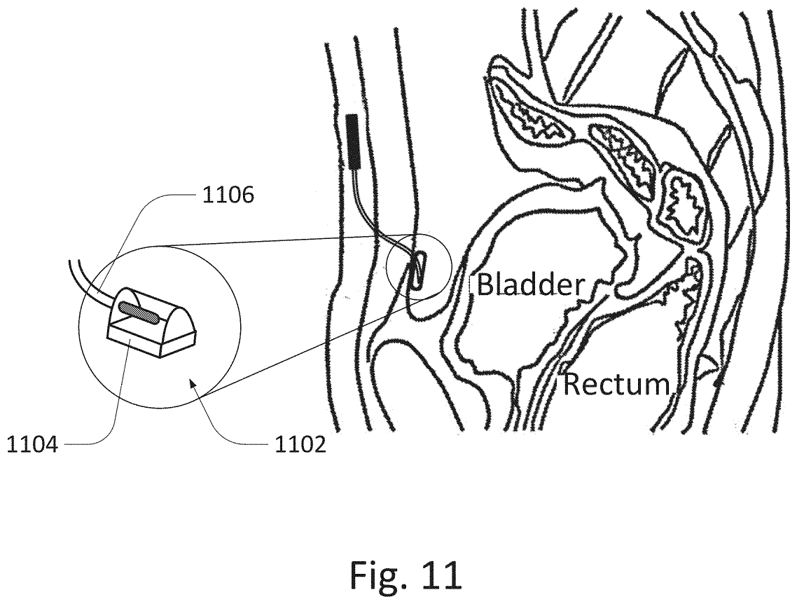

Sensor location within the body affects the glucose readings acquired by the sensor component. Placing a free floating sensor in the peritoneal cavity has proven to show good results, however a more precise location would potentially increase accuracy and repeatability. A more precise location also may make it easier to swap out a sensor after a certain amount of time (e.g. 18 months). It is desirable to place a replacement sensor approximately same location as the replaced sensor to achieve repeatable results. In the embodiment shown in FIG. 11, separately implantable “landing pad”, or base structure, 1102, is implanted into or onto a desired location of tissue (e.g., peritoneal lining, rectus sheath, etc) and becomes relatively permanently fixed to this region. The base structure may be manufactured all or in part from a material which allows tissue ingrowth (i.e. Dakron). For example, the base structure may include Dakron base component 1104. The base structure may have a collar, or marker, that a physician visualizes either by laparoscopy, x-ray, fluoroscopy, or ultrasound, so that he/she may guide sensor catheter assembly 1106 into or onto the base structure to fix the catheter in place.

The sensor component, in another embodiment, may be implanted in the fascia layer. Superficial, deep muscle, and visceral fascia are all possible sensor implantation sites. Tissue encapsulation of the sensor component may be less in these sites than in the peritoneal cavity. A glycol-enzymatic sensor, or other sensor types disclosed herein, may be used in this area. In these embodiments, sensing technology that allows analyte containing tissue to contact the sensing element are preferable.

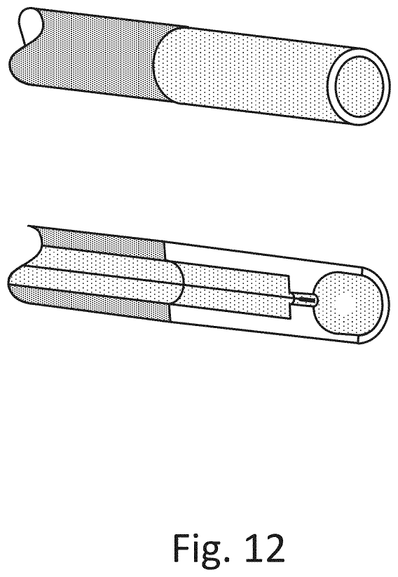

Varying analyte concentrations in the local milieu may possible lead to erratic sensor readings. Some embodiments of the sensor assembly component may include a shroud, or cover, over the sensor with a piston style actuation on the proximal end of the catheter and/or sensor assembly. The piston draws fluid into the chamber during a reading, and subsequently expels the fluid when the reading is complete. This piston action would occur every time a reading is necessary (e.g. once a minute), i.e., the mechanism would draw in and expel fluid. This would ensure fluid is being cycled through, or across, the sensor properly, so that no stagnant fluid is left in or on the sensor. See FIG. 12.

In another embodiment of the control portion of the system, peritoneal fluid is drawn into a reservoir that's fully internalized which contains a sensor or an array of sensors to measure the glucose concentration of the fluid in the reservoir, or passing to/from the reservoir. The fluid may then be expelled or it may be recirculated. This embodiment may increases uniformity of glucose concentration from sample to sample. This reservoir may be rigid, or flexible (e.g. a balloon). The reservoir may be externalized and attached to the outside of the patient or be implanted, for example in the preperitoneal or subcutaneous space. The fluid may be analyzed using mid-infrared or near-infrared spectroscopy or other wavelength spectroscopy.

Sensors based on chemistry that require a fluorescing dye experience a phenomenon known as photobleaching, which is defined as the photochemical destruction of the dye molecule. Due to this phenomenon, a sensor of this chemistry will operate for a finite amount of time that depends on the time of exposure to the excitation source (LED, e.g.). Therefore, despite flushing or anti-encapsulation mechanism described herein, the sensor may still fail to respond after a certain amount of time due to destruction of the dye molecule. To address this problem, several approaches may be used.

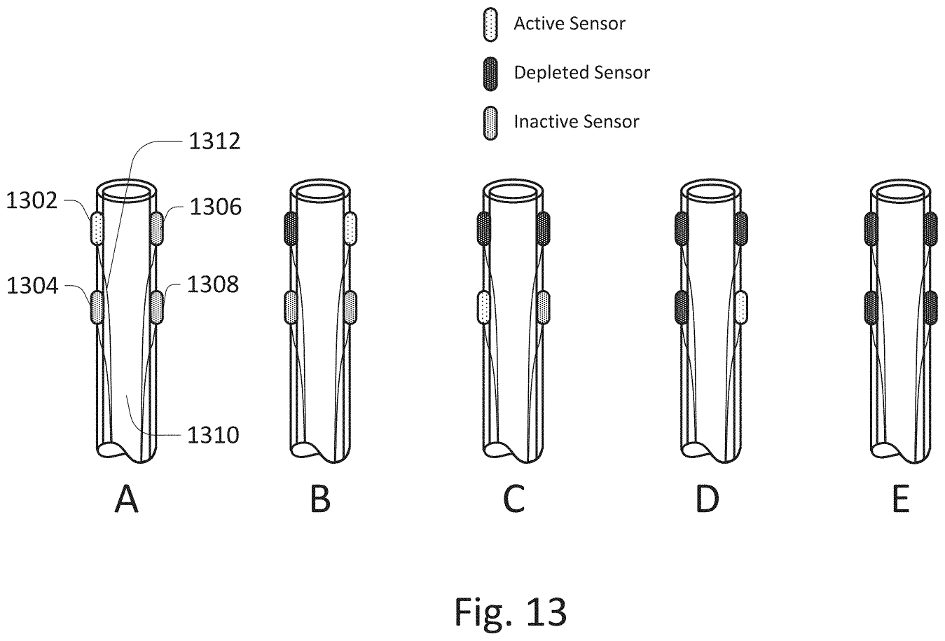

In the embodiment shown in FIG. 13, multiple sensors are incorporated into one tube/catheter assembly, each sensor with its own fiber optic cable, and each sensor having its own excitation source. The catheter/tube, in this embodiment and other embodiments, may be manufactured from any suitable biocompatible and flexible material, including silicone, polyvinyl chloride, polyurethane, etc. In this embodiment, a primary sensor may be excited by its excitation source while the other sensors remain dormant. After either a set period of time and/or a set degradation in signal strength, the primary excitation source may cease operating, and a second excitation source would become active, exciting a second sensor. This process continues until each of the sensors in the assembly has been chemically depleted, or a set amount of time has passed. FIG. 13 shows multiple sensors 1302, 1304, 1306 and 1308 on catheter 1310. Fiber optic cable 1312 is connected to sensor 1302. Additional fiber optic cables are connected to the other sensors. In step A, sensor 1302 is active and sensors 1304, 1306 and 1308 are inactive. Once sensor 1302 becomes depleted, as is shown in step B, sensor 1306 becomes active, while sensors 1308 and 1304 remain inactive. In step C, both sensors 1302 and 1306 are depleted, and sensor 1304 is now active, leaving sensor 1308 inactive. In Step D, sensors 1302, 1304 and 1306 are all depleted and sensor 1308 is active. Finally, in step E, all 4 sensors are depleted.

A variation on this embodiment would be to excite each sensor in turn, until the entire sensor assembly is chemically depleted, rather than fully depleting each sensor before moving on to the next sensor. For example, if there are 4 sensing elements in the assembly, and measurements were taken once a minute, the operation would be as follows:
Minute 1: Excite sensing element #1
Minute 2: Excite sensing element #2
Minute 3: Excite sensing element #3
Minute 4: Excite sensing element #4
Minute 5: Excite sensing element #1
Etc.

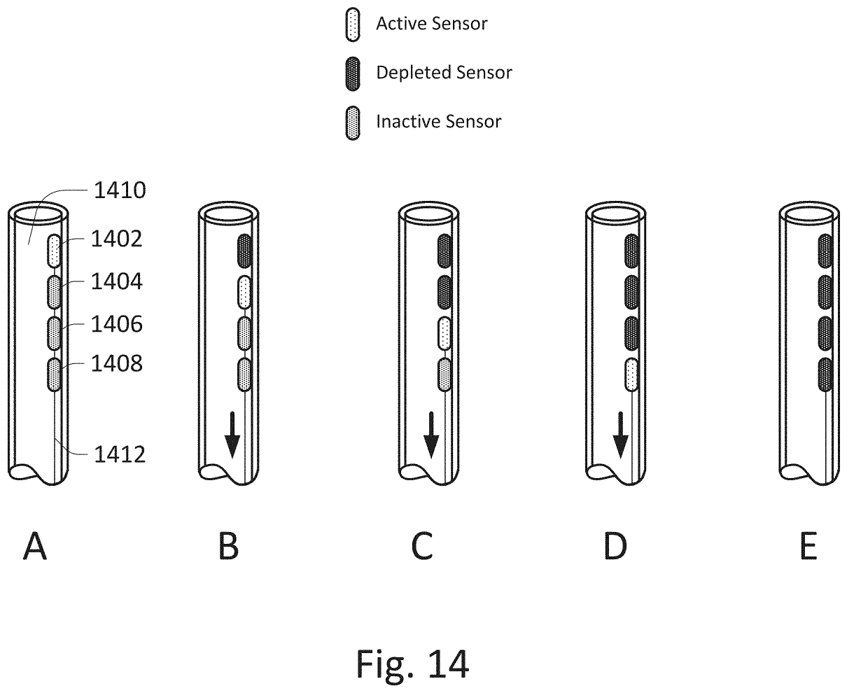

In the embodiment shown in FIG. 14, an array of sensing elements is incorporated into a catheter as in the embodiments described above. However, one excitation source for all sensing elements, rather than a separate excitation source for each sensing element is used. Sensors 1402, 1404, 1406 and 1408 are shown on catheter assembly 1410. The excitation source is in communication with fiber optic cable 1412. Fiber optic cable 1412 can be moved with respect to the catheter assembly, so that its most distal tip may be in communication with one sensor at a time, and then moved to excite another sensor. In step A, distal most sensor 1402 is in communication with fiber optic cable 1412 and is active. Step B shows the assembly after the signal has degraded on most distal sensor 1402 (or after a set time has expired). Excitation source cable or connector 1412 is pulled back proximally, so that its distal tip is aligned with the next most proximal sensing element, sensor 1404, which is now active. The fiber optic cable may be fixed in place until it is moved again. This process continues until each of the sensors in the assembly has been chemically depleted, or a set amount of time has passed, as is shown in subsequent steps C, D and E. This assembly may be linear or it may be coiled.

Any feature of any embodiment disclosed herein may be combined with other features and/or embodiments. For example, a flushing mechanism could be combined with a wiper collar mechanism to ensure encapsulation could be cleared. A mesh network may also incorporate encapsulation clearing features at one, or more, sensor element to ensure proper functionality.

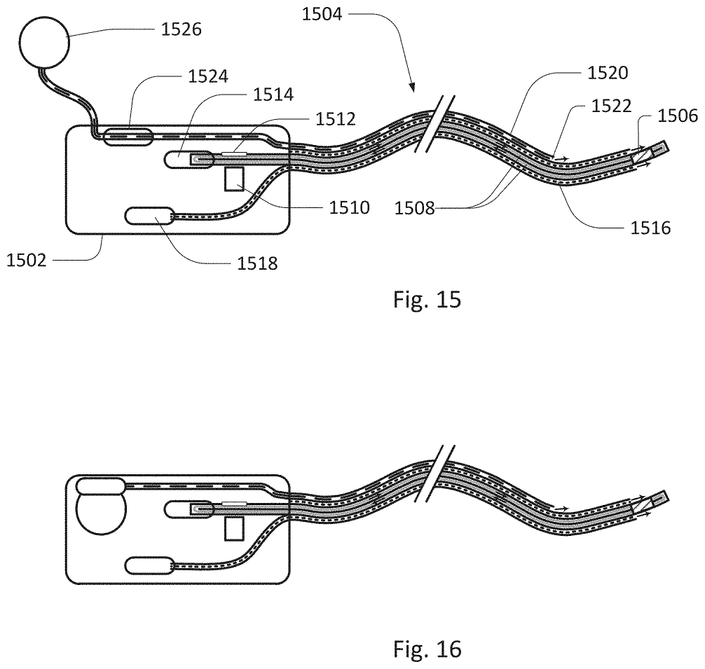

FIG. 15 shows an embodiment of the peritoneal sensor system which uses infrared, mid-infrared or near-infrared spectroscopy or other wavelength spectroscopy or spectrophotometry. Control portion 1502 is preferably implanted subcutaneously, but can be implanted anywhere as disclosed herein. The control portion may also be external as disclosed herein. catheter/sensor portion 1504 is preferably placed so that the distal tip is in the peritoneal cavity, however the catheter/sensor portion may be placed anywhere as disclosed herein. In this embodiment, the sensor portion is a sensor interface which includes micro-dialysis membrane 1506. The micro-dialysis membrane is permeable to certain analytes (for example, glucose), but is impermeable to contaminates.

The target analyte/glucose passively diffuses across the membrane, from the fluids in the peritoneal cavity, into the sampling fluid which is circulating within sampling lumens 1508. In this embodiment, the sampling fluid is continuously, or intermittently, circulated through sampling lumens 1508, preferably in a closed loop system, but the fluid may alternatively be circulated in an open loop system. The sampling fluid may also be referred to as the “perfusate” or “dialysate”. The sampling fluid may be sterilized and/or analyzed using light source/analyzer 1510 which shines light of particular wavelengths through the sampling lumen(s) and the sampling fluid to analyze the fluid for glucose content. Reflector 1512 may be utilized to reflect the light delivered from light source 1510 so that light source/analyzer 1510 may also be used to analyze the resulting light, after it has passed through the sampling fluid. Alternatively, the sampling fluid may be analyzed in a reservoir. The sampling fluid may alternatively include an antibiotic which is too large to pass through the membrane.

In this way, the sampling fluid is continuously, or intermittently, passing by micro-dialysis membrane 1506, where glucose diffuses into the sampling fluid, and the fluid containing the glucose is passed by light source/analyzer 1510 to collect data relating to glucose concentration. The glucose concentration within the sampling fluid reflects the glucose level within the patient. Sampling fluid pump 1514 may be used to circulate sampling fluid for analysis. UV light may also be delivered from light source/analyzer 1510 to sterilize the sampling fluid or UV light may be delivered from a separate source.

In preferred embodiments, sampling lumen(s) 1508 are small to allow for quicker response times to changing glucose levels in the body and also to minimize trauma to the body. For example, the ID of the sampling lumen may be from about 0.2 mm to about 0.5 mm. Alternatively, the ID of the sampling lumen may be from about 0.5 mm to about 1.0 mm.

In a closed loop system, the glucose concentration in the sampling fluid will equilibrate with the glucose concentration in the body and reflect changes to glucose concentration in the body quickly as a result.

The sampling lumens may be next to each other, or coaxial, so one inside the other. The micro dialysis membrane may be tubular, so wrapping around 360 degrees of the catheter assembly, or it may only cover a portion of the circumference of the catheter assembly.

The sampling fluid within the sampling lumen(s) may be under slightly negative pressure.

The peritoneal sensor system shown in FIG. 15 also includes flushing lumen 1516. The flushing lumen is used to flush tissue and/or other contaminates from micro-dialysis membrane 1506. Flushing lumen 1516 may be annular, so surrounding the sampling lumen(s) or may be of any other shape. Any other flushing or dislodging mechanism disclosed herein may also be used. Flushing fluid may be forced distally using flushing pump 1518, or by any other means. Flushing fluid may be saline, may come from a replenishable reservoir, for example a self-sealing injection port, and/or may be peritoneal fluid which is drawn into either the flushing lumen, or a flushing fluid reservoir from the peritoneal cavity. Flushing fluid may contain insulin. In embodiments where the flushing fluid is peritoneal fluid, the fluid may enter the flushing reservoir by a slow reversal of flushing pump 1518, or the fluid may be allowed to leak or seep into the reservoir over time. In this situation the reservoir, or a port to the reservoir, may be porous or perforated, or permeable.

Insulin delivery lumen 1520 ending in insulin delivery opening 1522 may also be incorporated into the system. Insulin pump 1524 may draw insulin from insulin reservoir 1526 and deliver it to the patient via insulin delivery lumen 1520 and out insulin delivery opening 1522. The insulin delivery opening may be distanced from micro-dialysis membrane 1506 to prevent glucose measurement interference caused by the added insulin. Insulin may be replenished in insulin reservoir 1526 by penetrating a self-sealing port within the reservoir with a needle through the skin of the patient. The insulin reservoir may be external to control portion 1502 or may be internal to the control portion. The flushing reservoir may be external to control portion 1502 or may be internal to the control portion.

Control portion 1502 includes a controller which regulates the various systems in communication with the control portion. These systems include the various pumps, the light wavelength analysis mechanism, control of the insulin delivery based on glucose measurements via the wavelength analysis mechanism, flushing, etc. Control portion 1502 may also include wireless communication technology (transmitter and/or receiver) to communicate with an external controller which may be a computer, mobile phone, tablet or other device. The external controller may include a wireless receiver/transmitter as well.

The external controller may include a display communicating the status of the various systems to the patient and/or his doctor. These displays may include glucose level, including glucose level over time, glucose level graph, glucose level averages, glucose level warnings, glucose level changes etc. The display may also include insulin delivery volumes, insulin delivery changes, insulin delivery alarms, insulin level within the system etc.

The control portion will also contain a battery, or other source of power, and may be replenished through an electromagnetic field, inductive charging etc.

Other embodiments include an agitation mechanism which agitates and/or vibrates the sensor/sampling component to help keep the area clean and free of ingrowth.

In some embodiments, peritoneal fluid is drawn into the catheter, or circulated through the catheter, and passes by the filter membrane. In these embodiments, the filter membrane may be inside the controller, or in the central or proximal areas of the catheter. The sampling catheter may have one, two or more lumens.

In moving continuous glucose monitoring to the IP space, we must also consider the properties of the new sensing environment which is known to have less access to fluids. In some embodiments, the sensor design includes the addition of a biocompatible polymer layer to the sensor to improve contact with local fluids, mitigate contact of hydrogen peroxide with local tissue, and provide an opportunity to manipulate the rate of diffusion of oxygen and glucose to the sensor chemistry for optimal signal by controlling the formulation, cross-linking, and thickness of the polymer. To improve signal integrity over time and protect tissue from hydrogen peroxide production of the glucose oxidase reaction in vivo, these embodiments includes the addition of polymer layer to the sensor surface. Biopolymers have tunable diffusive properties with respect to two inputs of the glucose sensing reaction —oxygen and glucose. A polymer layer can therefore be used to control reaction rates. Polymer cross-linking ratio and/or thickness tolerance requirements are optimized to obtain ideal diffusive properties for the physiological range of glucose of interest. Polymer layer assembly is dependent on the type of polymer formulation applied. Some possible polymers include alginate, polydimethylsiloxane (PDMS), and other hydrogels, medical coatings, or thin film used by the medical device industry. Assuming formulation is consistent, diffusion of analytes through a polymer will be highly dependent upon thickness. Some embodiments mitigate variation in polymer thicknesses during manufacturing by calibrating signal against background signal using redundant sensors. Including a polymer layer will also mitigate inaccuracies emerging from an IP-implanted sensor which is not continuously submerged in a homogeneous fluid to sense glucose concentrations.

Polymer coatings are preferably biocompatible, durable in vivo, hydrophilic, and permeable to oxygen and glucose. An example of such polymer are biocompatible hydrogels which are typically composed of a two components, a polymer and cross-linker, the latter which is activated upon exposure to UV radiation. Hydrogels are often mixed within a mold and cured to form desired shape. The ratio of polymer to cross-linker, and exposure to UV radiation, can be used to control the density and mechanical structure of hydrogel polymers.

In one embodiment, the sensor assembly consists of 3-4 modified sensors assembled into a dual-lumen tube within a silicone tube catheter. Wire sensors may be attached to the silver and platinum electrodes using conductive silver epoxy, and the joints may be encapsulated with insulating epoxy to prevent shorts due to fluid intrusion. Under clean conditions, sensors may be threaded down one lumen of the catheter. Proximal ends of the tubes are sealed and ports are bonded in line with each lumen to allow for flushing. The assembly may then be connected to the transmitter.

Lag times for blood glucose measurements in the IP space using the IP continuous glucose monitor may be less than around 12 min. Alternatively the lag times for blood glucose measurements in the IP space using the IP continuous glucose monitor may be less than around 10 min. Alternatively the lag times for blood glucose measurements in the IP space using the IP continuous glucose monitor may be less than around 7 min. Alternatively the lag times for blood glucose measurements in the IP space using the IP continuous glucose monitor may be less than around 5 min.

In some embodiments, redundant sensors for glucose signal, background signal, and oxygen signal are included. Redundancies in sensors provides the algorithms with the data points required for calibration of glucose signal against background signal and oxygen tension during physiologic changes in the local environment. Calibration against background is well known in signal processing to improve accuracy and reduce noise of the target signal. It has been shown that adding redundant and reference sensors to a single device can improve accuracy by providing larger sample size over which signal can be averaged, a reference signal on background, and a reference signal on local fluctuations on environmental factors such perfusion, oxygenation, and encapsulation. One role of an oxygen sensor is to improve accuracy of glucose estimates by allowing algorithms to correct for small changes in oxygen tension which occur during physiologic changes. Another role of an oxygen sensor is to enable early-warning of peritonitis, the consequences of peritonitis are potentially severe, especially in immune compromised T1 OM patients. To mitigate this risk, the IP CGM device includes a proprietary algorithm for use in implantable devices which provides early indication of an infection based on combined oxygen/glucose sensor readings.

Some embodiments include a wearable receiver display in wireless communication with a fully implanted sensor and transmitter.

Example of Data Processing System

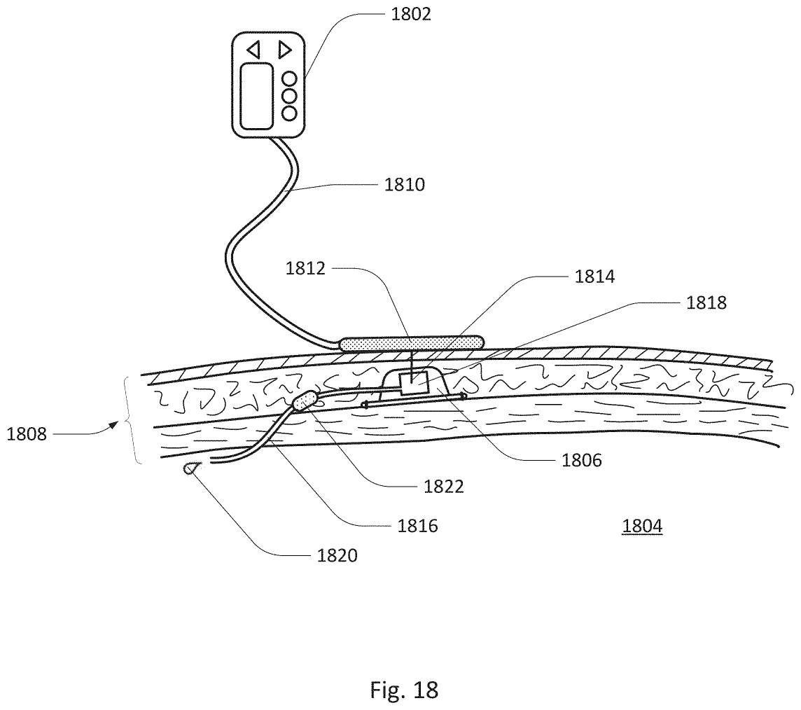

FIG. 17 is a block diagram of a data processing system, which may be used with any embodiment of the invention. For example, the system 1700 may be used as part of the control component of the peritoneal sensing system. Note that while FIG. 17 illustrates various components of a computer system, it is not intended to represent any particular architecture or manner of interconnecting the components; as such details are not germane to the present invention. It will also be appreciated that network computers, handheld computers, mobile devices, tablets, cell phones and other data processing systems which have fewer components or perhaps more components may also be used with the present invention.

As shown in FIG. 17, the computer system 1700, which is a form of a data processing system, includes a bus or interconnect 1702 which is coupled to one or more microprocessors 1703 and a ROM 1707, a volatile RAM 1705, and a non-volatile memory 1706. The microprocessor 1703 is coupled to cache memory 1704. The bus 1702 interconnects these various components together and also interconnects these components 1703, 1707, 1705, and 1706 to a display controller and display device 1708, as well as to input/output (I/O) devices 1710, which may be mice, keyboards, modems, network interfaces, printers, and other devices which are well-known in the art.

Typically, the input/output devices 1710 are coupled to the system through input/output controllers 1709. The volatile RAM 1705 is typically implemented as dynamic RAM (DRAM) which requires power continuously in order to refresh or maintain the data in the memory. The non-volatile memory 1706 is typically a magnetic hard drive, a magnetic optical drive, an optical drive, or a DVD RAM or other type of memory system which maintains data even after power is removed from the system. Typically, the non-volatile memory will also be a random access memory, although this is not required.

While FIG. 17 shows that the non-volatile memory is a local device coupled directly to the rest of the components in the data processing system, the present invention may utilize a non-volatile memory which is remote from the system; such as, a network storage device which is coupled to the data processing system through a network interface such as a modem or Ethernet interface. The bus 1702 may include one or more buses connected to each other through various bridges, controllers, and/or adapters, as is well-known in the art. In one embodiment, the I/O controller 1709 includes a USB (Universal Serial Bus) adapter for controlling USB peripherals. Alternatively, I/O controller 1709 may include IEEE-1394 adapter, also known as FireWire adapter, for controlling FireWire devices, SPI (serial peripheral interface), I2C (inter-integrated circuit) or UART (universal asynchronous receiver/transmitter), or any other suitable technology.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques shown in the figures can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals).

The processes or methods depicted in the preceding figures may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

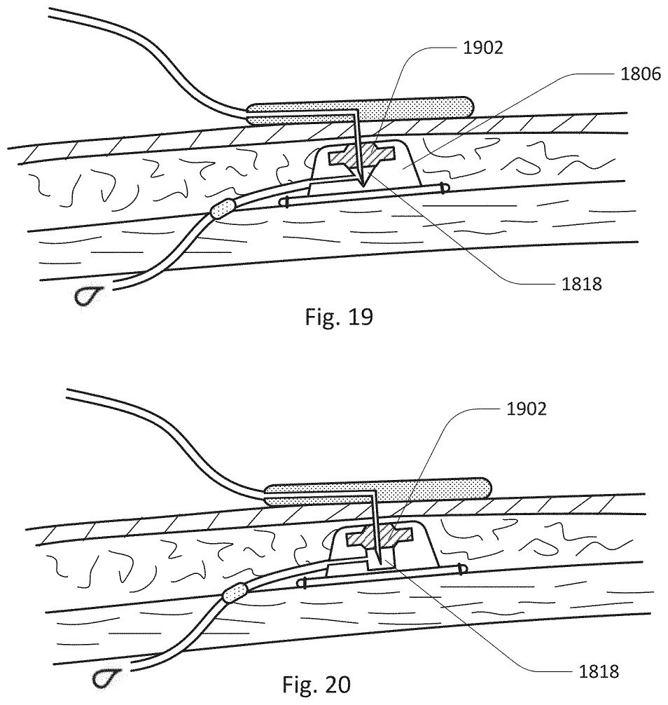

FIG. 18 shows an embodiment of a peritoneal insulin delivery system. In this embodiment, insulin pump 1802 pumps insulin into peritoneal cavity 1804. Subcutaneous port 1806 is implanted in or under skin layer 1808, which includes skin, fat and muscle. Insulin pump 1802 is fluidly connected to infusion tubing 1810 which can be connected to patch 1812 which may be adhered to the outside of the skin. Patch 1812 includes needle or cannula or access device 1814 which pierces the skin and enters subcutaneous port 1806. Needle 1814 is in fluid communication with tubing 1810. The patch with needle 1814 may be removed and replaced with a different patch/needle, while the subcutaneous port is designed to remain implanted for a longer period of time, for example 1-3 months, or 3-6 months, or 6-12 months, or more than 1 year.

The insulin pump is meant to be worn on the subject's body/clothing so that the subject may be ambulatory. The pump is generally meant to be worn at all times, except when the system is being replaced, flushed, etc.

Subcutaneous port 1806 includes reservoir 1818 which is in fluid communication with needle 1814 and implanted insulin delivery tubing 1816. Delivery tubing may include Dacron cuff 1822. In this way, insulin 1820 may be delivered to peritoneal cavity 1804 from insulin pump 1802.

Preferably, needle 1814 is a non-coring needle, such as a Huber needle. In some embodiments the needle may have more than one outlet port so that the likelihood of it being blocked is reduced.

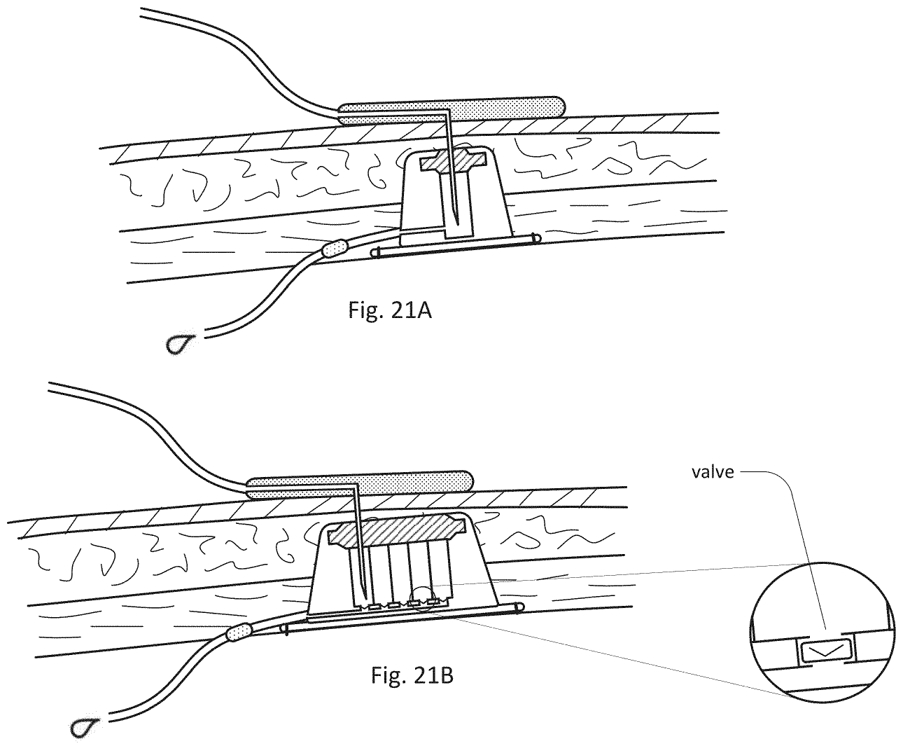

FIG. 19 shows more detail of subcutaneous port 1806. The subcutaneous port includes piercable septum 1902 and reservoir 1818. The septum may be made out of silicone rubber, other rubber, or other suitable material. Preferably, the septum “reseals” after a needle/cannula has been removed. It is also preferable that the septum be able to be pierced multiple times and possibly in multiple locations and at different angles.

Reservoir 1818 may be of various sizes and shapes. Shown in FIG. 19 is a tapered reservoir which helps guide the needle tip into the base of the reservoir. The angle of the tapered reservoir may be different than the angle of the needle so that the hole of the needle does not get blocked by the wall of the reservoir. For example, the angle of the needle tip may be less than the angle of the taper of the wall. Alternatively, the angle of the needle tip may be greater than the angle of the taper of the wall of the reservoir.

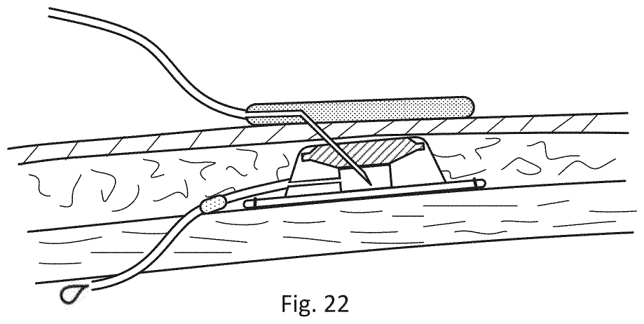

FIG. 20 shows a larger, cylindrical shaped reservoir.

The volume of the reservoir may be designed to be as small as possible to minimize the volume of insulin that remains in the reservoir after insulin pumping. For example, the volume of the reservoir may be about 1-10 mm3. Alternatively, the volume of the reservoir may be about 10-50 mm3. Alternatively, the volume of the reservoir may be about 50-100 mm3. Alternatively, the volume of the reservoir may be about 100-1000 mm3. Alternatively, the volume of the reservoir may be about 1000-10000 mm3.

Alternatively, the volume of the reservoir may be designed with a precise and known volume so that insulin delivery volumes may be calibrated to compensate for any stagnant insulin volume in the reservoir. Alternatively, a flushing mechanism may be used to flush the reservoir of any insulin when a precise volume of insulin is pumped from the insulin pump into the peritoneal cavity.

Since the patch is replaceable, a “docking” or matching system may be useful to align the needle of the patch with the septum of the subcutaneous port so that when a new patch is applied to the skin, the needle of the patch is sure to pierce the septum of the port and enter the reservoir of the port. The docking system may include magnetics, so that the patch is automatically centered or aligned with the subcutaneous port before the needle is deployed into the port. Alternative or additionally, bumps, or ridges or other tactile features may be included on the skin-facing side of the port so that they may be felt through the skin to aid in alignment.

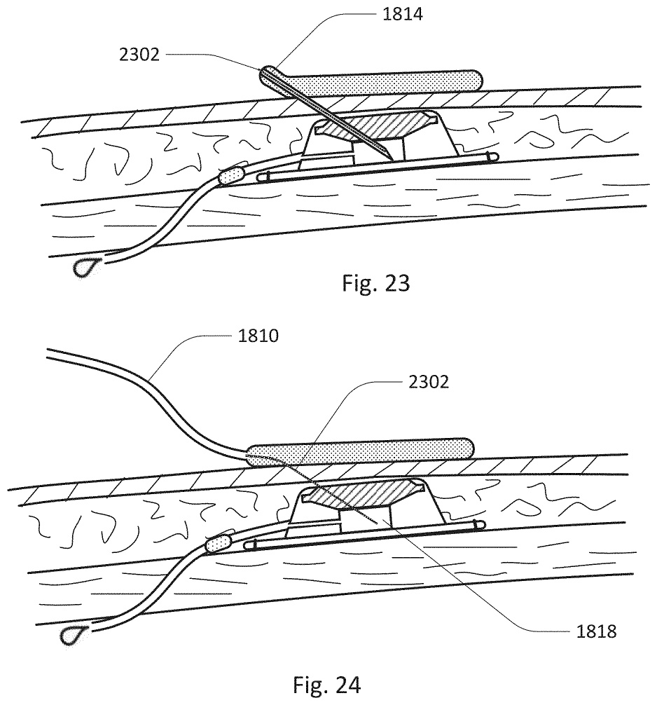

FIGS. 21A and 21B show a subcutaneous port with a deeper reservoir which ensures that the tip of the needle will reside in the reservoir after the patch has been secured to the skin. This deeper reservoir allows for more variation in the depth of the port implantation. For example, the reservoir may be 1-5 mm deep, alternatively the depth of the reservoir may be 10-15 mm, alternatively the depth of the reservoir may be 5-10 mm, alternatively the depth of the reservoir may be 15-20 mm.

Some embodiments may include a mechanism which ensures the implantation of the port is performed at a precise depth.

Some embodiments may include a registration mechanism in the port (and/or the needle) which captures the tip of the needle, or prevents the tip of the needle from advancing further or prevents the needle from withdrawing before the user is ready to remove the patch. Some embodiments may include a lock, such as a ball/socket or twist lock. The lock may include a magnetic component. The lock may provide feedback to the user when the needle is correctly placed within the reservoir of the subcutaneous port. For example, there may be audible, hepatic, visual or other feedback.

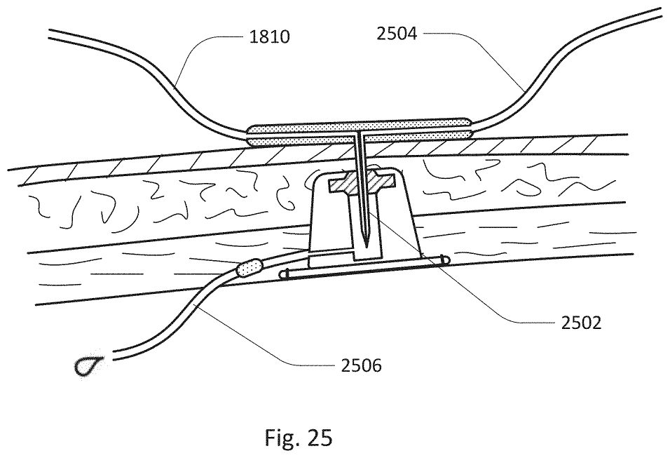

FIG. 22 shows a subcutaneous port with a septum with a larger surface area and possibly a reservoir with a larger area interface with the septum. The patch shown in FIG. 22 also includes an angled needle, although a straight needle, as shown in FIGS. 21A and 21B, may also be used. The advantage of using an angled needle is that when a new patch is applied to the skin, the needle may be positioned in a slightly different orientation than the previous times which avoids piercing the septum in the same place. In this embodiment, the surface area of the septum may be around 100 mm2 to 150 mm2. Alternatively, the surface area of the septum may be around 150 mm2 to 200 mm2. Alternatively, the surface area of the septum may be around 200 mm2 to 300 mm2. Alternatively, the surface area of the septum may be around 300 mm2 to 400 mm2. Alternatively, the surface area of the septum may be around 400 mm2 to 500 mm2. Alternatively, the surface area of the septum may be around 500 mm2 to 1000 mm2. Alternatively, the surface area of the septum may be over 1000 mm2.

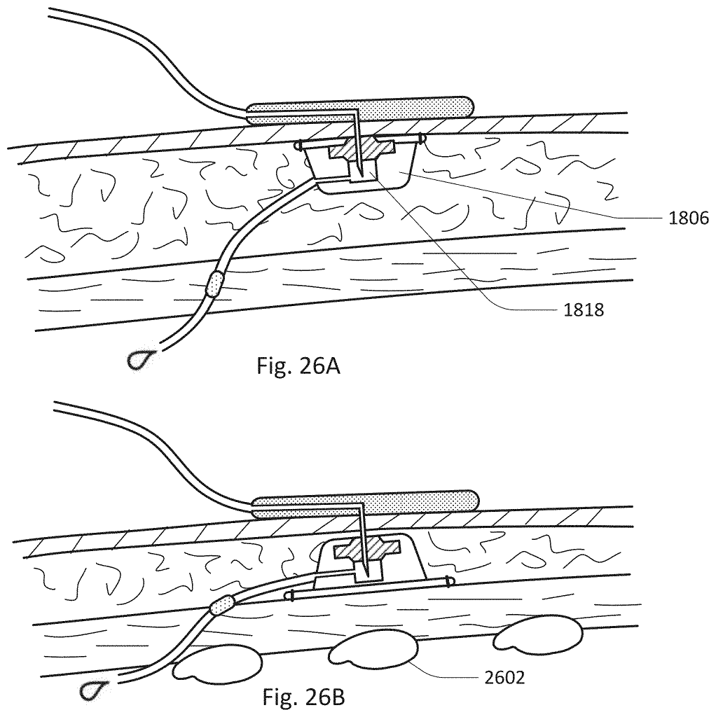

FIGS. 23 and 24 show an embodiment of the insulin delivery system where needle 1814 is removed after the patch has been placed on the skin. In this embodiment, needle 1814 is removed, and cannula 2302 is left behind. Cannula 2302 is in fluid communication with infusion tubing 1810 and reservoir 1818. Having a cannula remain in place instead of a needle may minimize discomfort for the user, as well as ongoing damage to the septum and subcutaneous port caused by body movement over time. The cannula may be made from any appropriate material, including polyethylene, silicone, polyimide, polymer, etc. The cannula may be reinforced, for example with a metal braid within the wall, on the interior or the exterior of the cannula to prevent kinking. The cannula may be designed to be bent/angled, without kinking, so that it can accommodate different fat/skin thicknesses.

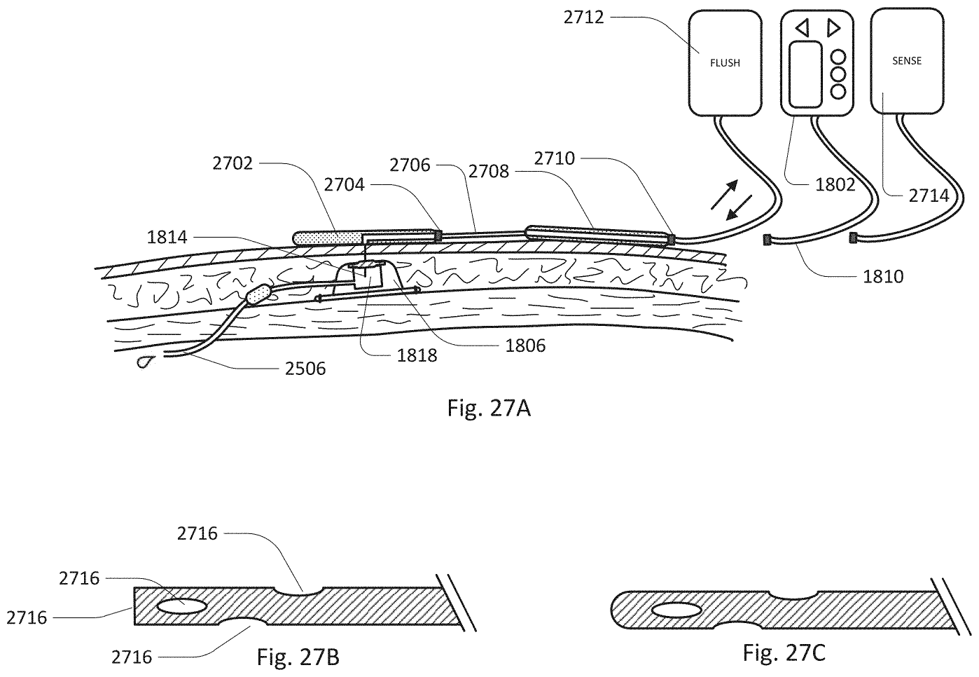

FIG. 25 shows an embodiment which combines insulin delivery and peritoneal cavity sensing, such as glucose sensing. In this embodiment a double lumen needle 2502, or 2 separate needles may be used. The needle lumens may enter the same reservoir (as shown here), or 2 separate reservoirs. In the case of a shared reservoir, tubing/catheter 2506 may be used for both insulin delivery and peritoneal cavity fluid sampling to determine glucose levels. Alternatively, 2 different catheters may be used. The lumen of infusion tubing 1810 is in fluid communication with one of the needle lumens, and sampling tubing 2504 is in fluid communication with the second needle lumen. Sampling tubing 2504 is in fluid communication with a peritoneal sensor system (not shown) to sense the amount of glucose in the sample from the peritoneal cavity. Alternatively the sensor system may be implanted.

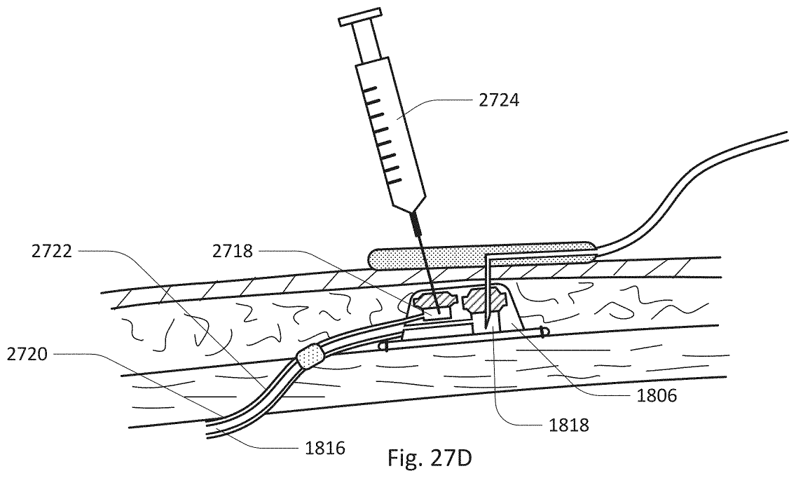

FIGS. 26A and B show embodiments of the insulin delivery system which may be particularly useful with overweight patients, but may be used on any patients. In patients where the fat layer is thicker than normal, as shown in FIG. 26A, subcutaneous port 1806 may be attached to the under layer of the skin (dermis), with sutures, glue, staples or other suitable mechanism. In this way, the location of reservoir 1818 with respect to the outer surface of the skin is relatively small and predictable. FIG. 26B shows an embodiment of the insulin delivery system where the subcutaneous port is designed to be implanted near ribs 2602. The subcutaneous port may be secured to at least one rib, or may be secured near the rib or ribs, using the rib(s) for stability. Generally, a patient has less fat near the ribs as well.

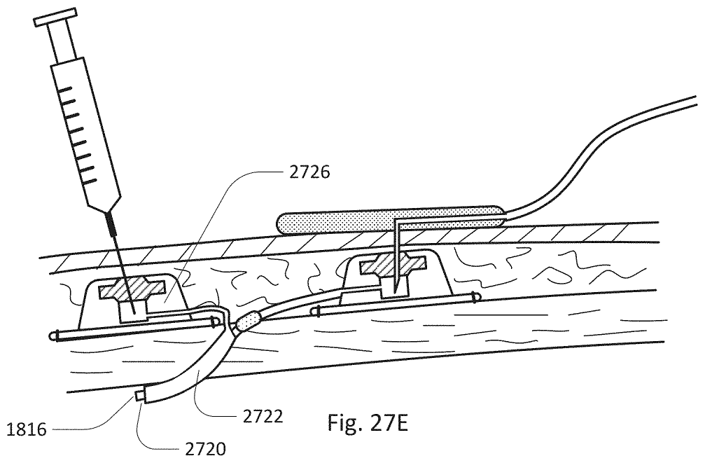

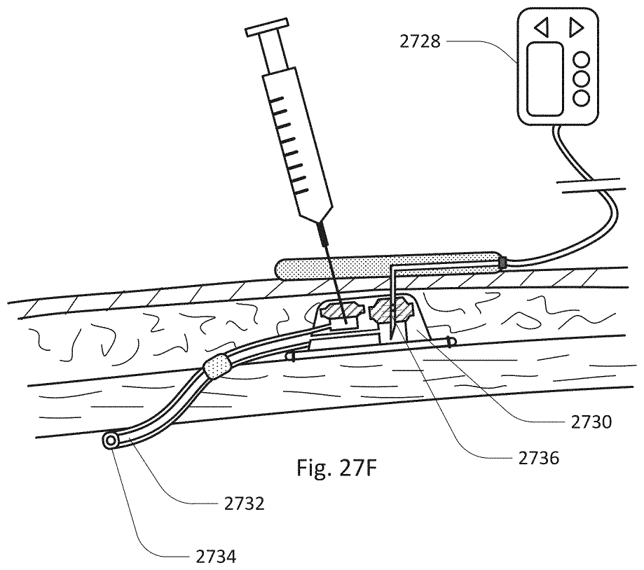

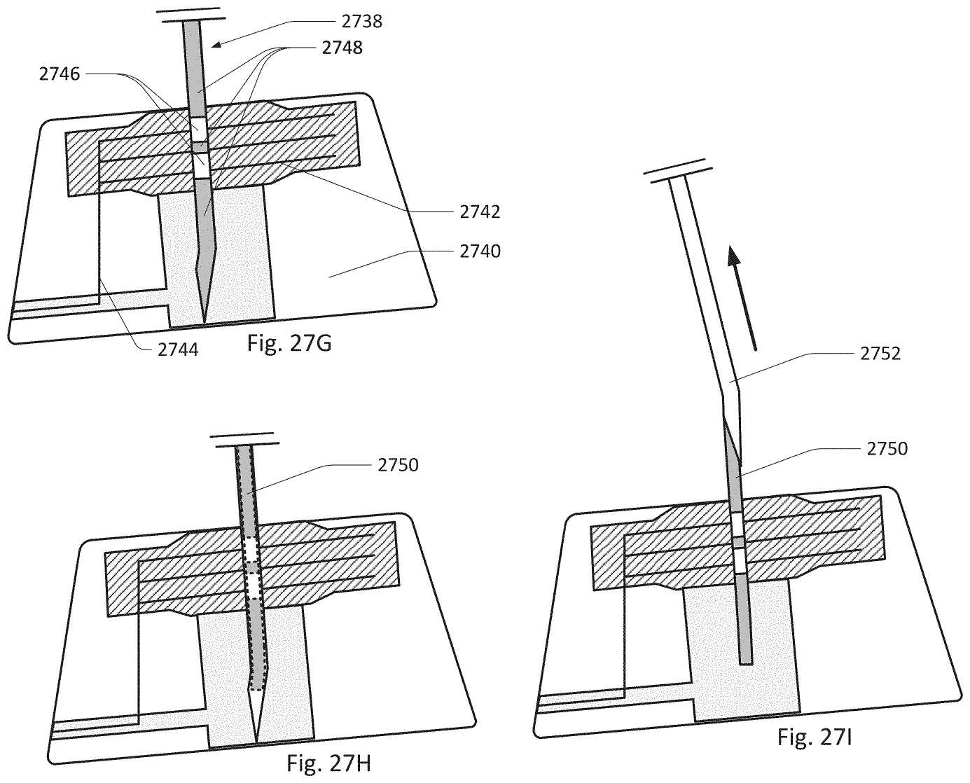

FIGS. 27A-C show embodiments of the insulin delivery system which may incorporate flushing of the system, as well as analyte sensing of fluid within the peritoneal cavity. This embodiment includes patch 2702 over subcutaneous port 1806. Patch 2702 is similar to patch 1812 disclosed in other embodiments disclosed herein. Patch 2702 may include connector 2704 to connect to connector tubing 2706 which connects to supplemental patch 2708 which may also be affixed to the skin of the subject/patient. Connector 2710 may connect to insulin pump 1802, flushing mechanism 2712, sensing mechanism 2714, or a device that include two or all of the pumping, flushing and sensing functions.

Insulin pump 1802 may be connected to the system to deliver insulin into the peritoneal cavity as disclosed elsewhere herein.

Occasionally, the system, and especially catheter/tubing 2506, may need to be flushed to prevent and/or eliminate clogs. Generally it is undesirable to flush all the insulin residing in reservoir 1818 into the peritoneal cavity to flush the system, because of the physiological effects. Embodiments that include a flushing mechanism may include a mechanism to first pull fluid into tubing/catheter 2506 from the peritoneal cavity. The peritoneal fluid displaces the insulin inside tubing/catheter 2506, reservoir 1818, needle 1814, patch 2702, connector tubing 2706, and supplemental patch 2708. After the insulin in these components has been replaced with peritoneal fluid, the system may be flushed with saline or other appropriate fluid. Flushing mechanism 2712, for this reason, may have the ability to pull suction (apply negative pressure) and also apply positive pressure to the system.

Sensing system 2714 may be incorporated into the flushing system and/or the insulin pump. Sensing system includes an analyte sensor, such as a glucose sensor, to sense the analyte/glucose in the peritoneal fluid. This sensing may occur in conjunction with the flushing process, after the peritoneal fluid has been drawn into the system.

Flushing system 2712 and sensing system 2714 may be incorporated into one system. They may also each or both be incorporated into a single system with the insulin pump.

Before insulin delivery can be initiated again after flushing the system and/or sensing an analyte in the peritoneal fluid, or when the insulin pump is first connected, the system may need to be primed with insulin. This will allow any incremental volume of insulin pumped from the pump into the system to enter the peritoneal cavity. One method of priming the system is to pump precisely the volume of insulin necessary to fill the system. This would include the volume inside tubing/catheter 2506, reservoir 1818, needle 1814, patch 2702, connector tubing 2706, and supplemental patch 2708. This may also include the volume inside infusion tubing 1810 connected to the infusion pump. Or, alternatively, tubing 1810 may not need to be primed if it is still full of insulin from when it was disconnected from the system.

A supplemental patch is shown here to increase the convenience of making repeated connections. In this way, any connections (to flush, sense, deliver insulin, bath, wash etc.) can be made at connection point 2710 instead of connection point 2704. This helps protect patch 2702 from repeat stresses that may loosen or adversely affect the sterile connection through the skin via needle 1814, allowing patch 2702 to stay in place for a longer period of time. Alternatively, supplemental patch 2708 may not be present and the various systems (pump, flushing mechanism, sensing mechanism) may be connected via connector 2704.

Alternatively, flushing of the system can be performed by monitoring the volume of insulin in the system (via the controller), and when the flushing time approaches, begin infusing flushing solution instead of insulin when insulin dosing is required. The flushing solution will push the insulin out of the system incrementally with each infusion at an approximately 1:1 volume ratio. So, for example, if 0.15 ml of insulin is required, approximately 0.15 ml of flushing solution will be infused into the system to push out a corresponding 0.15 ml of insulin. When the insulin in the system is depleted, or nearly depleted, the system may then initiate a flush sequence to remove any blockages in the system. The advantage to this approach is that insulin does not need to be removed from the system using a separate mechanism in order to flush the system. Depending on the volume of the system, the introduction of flushing solution instead of insulin may depend hours or even days before the actual flush sequence is performed.

Alternatively, flushing of catheter 1816 may be performed via a different lumen than the insulin lumen. For example, as shown in FIG. 27D, port 1806 may include more than one reservoir, one for insulin, insulin reservoir 1818, and one for flushing solution, flushing reservoir 2718. The reservoir for flushing solution is in fluid communication with flushing lumen 2720 of catheter 1816, shown here as defined by sheath 2722. Flushing lumen 2720 may alternatively not be part of catheter 1816. The flushing lumen may be concentric with (i.e. outside of, as shown here) or next to, the insulin lumen of catheter 1816. The flushing reservoir in the port may be accessed directly with syringe 2724, as shown here, or may be in fluid communication with a flushing fluid pump external to the body and preferably controlled by the controller.

Alternatively, a second reservoir/port for flushing may be implanted and in fluid communication with the flushing lumen of the catheter, as shown in FIG. 27E. Here, flushing port 2726 contains the flushing reservoir and can be accessed via a syringe, as shown here, or via a pump.

The distal end of the flushing lumen may be flush with, distal to, or proximal to the distal end of catheter 1816.

In some embodiments, insulin may be used to flush the catheter. This would need to be achieved with very small volumes of insulin, requiring a very small opening of the insulin lumen of the catheter to achieve adequate pressure/flow to flush the tip of the catheter.

In some embodiments the distal tip of the catheter may be cleaned by occasionally physically collapsing the distal end of the catheter, breaking the bond with any adhesions. This may be done by occasionally applying a negative pressure to the distal end of the catheter. This may be done by pulling a vacuum on the system via the controller.

FIGS. 27B and 27C show possible embodiments of the tip of tubing/catheter 2506 which allow peritoneal fluid to be withdrawn into the catheter/tubing from the peritoneum. Openings 2716, which may be arranged around the distal tip of the tubing/catheter so that at least one will remain open regardless of the position of the tubing/catheter within the peritoneal cavity. The catheter/tubing may include a distal opening as well, as shown in FIG. 27B, or have a closed, possibly rounded distal end, as shown in FIG. 27C.

FIG. 27F shows an embodiment of the system which includes both insulin (or other fluid) injection and glucose (or other analyte) sensing. In this embodiment, controller 2728 may perform both the insulin injection and glucose monitoring function, or the functions may be separate. Controller 2728 may be closed-loop or semi closed-loop. For example, the injection of insulin may be solely driven by the sensed glucose levels, or may be partially driven by the sensed glucose levels. The user may be able to override or augment the insulin delivery based on other factors, such as how the user feels, or what the user eats. The closed-loop function may make only small adjustments to insulin levels and not large adjustments, or may make all or most adjustments automatically.

Glucose sensing may be performed by drawing peritoneal fluid from within the peritoneal cavity either into subcutaneous port 2730 or into controller 2728, or elsewhere outside of the body, where a glucose sensor senses the glucose within the fluid. Alternatively, glucose sensor 2734 may be on catheter 2732 as shown here. In the embodiments where the glucose sensor is incorporated into the catheter, or the port, an electrical connection between the controller and the sensor may be required. This may be achieved via smart needle 2736, or may be achieved wirelessly or by other mechanisms.

FIGS. 27G-27I show various embodiments of the system which includes a smart needle, or a smart needle/cannula which achieves an electrical connection between the controller and an implanted sensor. In addition, the smart needle/cannula may have the ability to infuse a fluid. Both the electrical connection and the infusion may be achieved by inserting the smart needle/cannula into a smart subcutaneous port. Some embodiments achieve this with a single stick, via a single needle or a single needle/cannula combination.

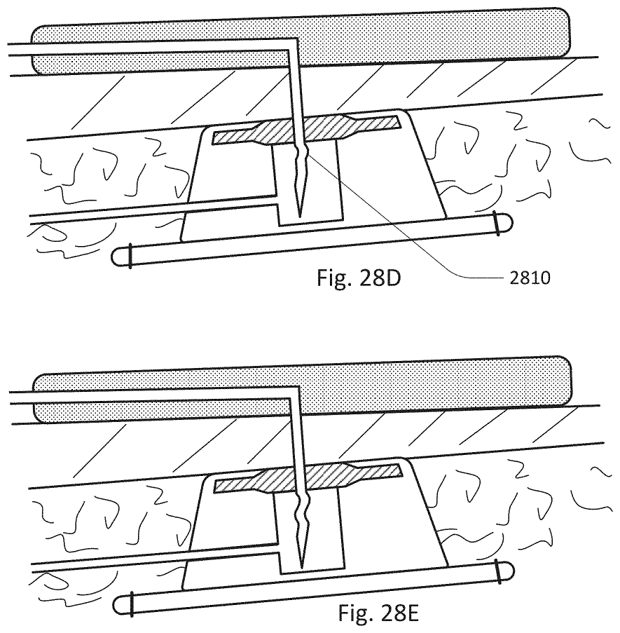

FIG. 27G shows an embodiment of a smart needle system which includes smart needle 2738 and smart subcutaneous port 2740. In this embodiment, the smart subcutaneous port includes multiple conductive mesh sheets 2742. These mesh sheets may be similar to those disclosed in FIG. 29B. These mesh sheets are connected to electrical connector 2744 which is in electrical communication with a sensor, such as a glucose sensor, on the catheter, or elsewhere in the implanted system. Needle 2738 includes conductive portions 2746 and insulated portions 2748. The distal and proximal insulated portions may extend to the ends of the needle, or only insulate a portion of the proximal and distal ends of the needle. The conductive portions of the needle are in electrical communication with the controller.

The mesh sheets in this embodiment are conductive, so that when a conductive portion of the needle is in physical contact with one of the conductive mesh sheets, the controller is in electrical communication with the sensor on the catheter or elsewhere in the system. One, two, or more electrical contacts may be present between the controller and the sensor, via the needle and the conductive mesh sheets. Two electrical connections are shown here.

The conductive mesh sheets and conductive areas on the needle may be arranged so that one, two, or another number of electrical contacts can be made at different needle depths, for example, with patients with different fat layer thicknesses. The smart needle may have more conductive surfaces than the number of conductive mesh sheets in the port, or the smart needle may have fewer conductive surfaces than the number of conductive mesh sheets in the port, as shown here.

FIGS. 27H and 27I show an embodiment of a smart needle which includes a smart cannula. In this embodiment, needle 2752 contains (or is contained by) cannula 2750. FIG. 27H shows the smart cannula within the needle as the needle is inserted into the smart subcutaneous port. In this embodiment, the cannula includes conductive and insulated portions. The needle may also include conductive and insulated portions, or it may not. To place the cannula, the needle is either inserted a set distance, or as far as it can be so that it bottoms out. Alternatively the needle is inserted past a set point, to make sure that the needle is in the reservoir of the port. The needle is then removed, while the cannula remains in place, as shown in FIG. 27I. The cannula may be pulled back slowly until the conductive portion(s) of the cannula is in contact with the conductive mesh(es) of the smart port. The smart cannula may be flexible or rigid, made out of any suitable material such as metal, or polymer.

One of the conductive portions of a smart needle or smart cannula may serve as a ground electrode.

Conductive meshes are shown here, but other electrical contacts are envisioned, including nodes, surfaces, protrusions etc. within the reservoir of the port.

In some embodiments, the controller can identify when the smart needle/cannula is in the desired position in the smart port, with one or two or more conductive portions of the smart needle/cannula in electrical contact with one or two or more conductive portions (i.e. conductive mesh) of the port. For example, the controller may analyze the resistance or conductivity between two electrodes on the smart needle/cannula to determine if the smart needle/cannula is in air, in skin, in fat, in other tissue, in the incorrect position within the port, or in the correct position within the port. The conductivity/resistance between two conductive portions (also referred to as electrodes) may be as follows:
The smart needle/cannula is in air—the resistance may be very high, or the conductivity low.
The smart needle/cannula is correctly positioned in the port—the conductivity/resistance may be set to be a specific value, so that this condition is easily identified.
The smart needle/cannula is in skin or fat or other tissue, or incorrectly positioned in the port—the resistance/conductivity may be different than that when the smart needle/cannula is in air or correctly positioned in the port.

The mesh may be flat, or curved. The mesh may be parallel to the surface of the port or may be at an angle to the surface of the port.

The smart needle or smart cannula may be hollow, as shown here, or may be solid.

In any of the embodiments disclosed herein, the sensing of glucose and/or delivery of insulin, may be subcutaneous, rather than in the peritoneal cavity.

Any of the embodiments disclosed herein may include one or more of the needle capture features disclosed herein and may include a mesh as part of the piercable septum and/or the subcutaneous port.

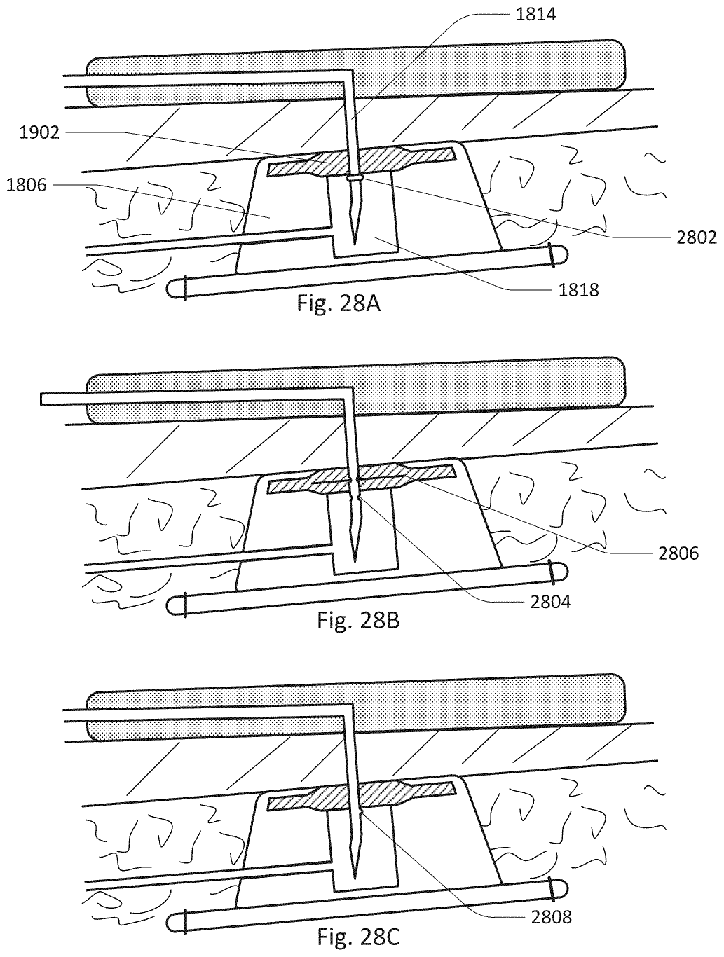

FIGS. 28A-E show various embodiments of the insulin delivery system which include features to help maintain needle 1814 within reservoir 1818 of subcutaneous port 1806. FIG. 28A shows an embodiment where needle 1814 includes small annular protuberance 2802 which is small enough to allow the needle to pierce the skin and piercable septum 1902, but is large enough that the needle does not easily slide out of its position within the subcutaneous port because the piercable septum “seals” around the smaller part of the needle. The protuberance may be on the outside of the needle, or may be constructed into the wall of the needle itself. In other words, the ID of the needle may or may not include an inverse protuberance. Embodiments may include one or more small annular protuberances.

FIG. 28B shows an embodiment where the needle includes small annular notch or indent 2804, similar to that of a post-style earring. In this embodiment the septum may include a plate, or mesh 2806, either embedded in the septum, or on either side of the septum (preferably the reservoir side). The mesh may help prevent the needle from easily slipping out of the reservoir, in the same way a post-style earring back prevents an earring from coming out of an earlobe. More details on possible mesh embodiments are disclosed in FIGS. 29A-C. The indent may be on the outside of the needle, or may be constructed into the wall of the needle itself. In other words, the ID of the needle may or may not include an inverse indent. Embodiments may include one or more small annular notches.

FIG. 28C shows an embodiment where the needle includes small non-annular protuberance 2808. This may be a simple “blob” or bump on one side of the needle. The bump may be on the outside of the needle, or may be constructed into the wall of the needle itself. In other words, the ID of the needle may or may not include an inverse protuberance. Embodiments may include one or more bumps.

FIG. 28D shows an embodiment where the needle includes irregularity, bend, or curvature 2810. This curvature is preferably constructed into the wall of the needle itself, for example, by adding a bend to a straight needle. Embodiments may include one or more bends, for example, see FIG. 28E for a needle with multiple bends.

Any of the needle feature embodiments may be used with a mesh. FIG. 29A shows an embodiment with mesh 2806 incorporated into the reservoir side of septum 1902. FIG. 29B shows an embodiment with multiple meshes. Note that the mesh(es) may or may not extend to the edges of the septum. The mesh(es) are generally large enough to cover the area of the top of reservoir 1818.

FIG. 29C shows some examples of mesh designs. The openings in the mesh may be regular or irregular, larger or smaller than the needle. The mesh may be made out of metal, polymer, wire, foam, or any other suitable material.

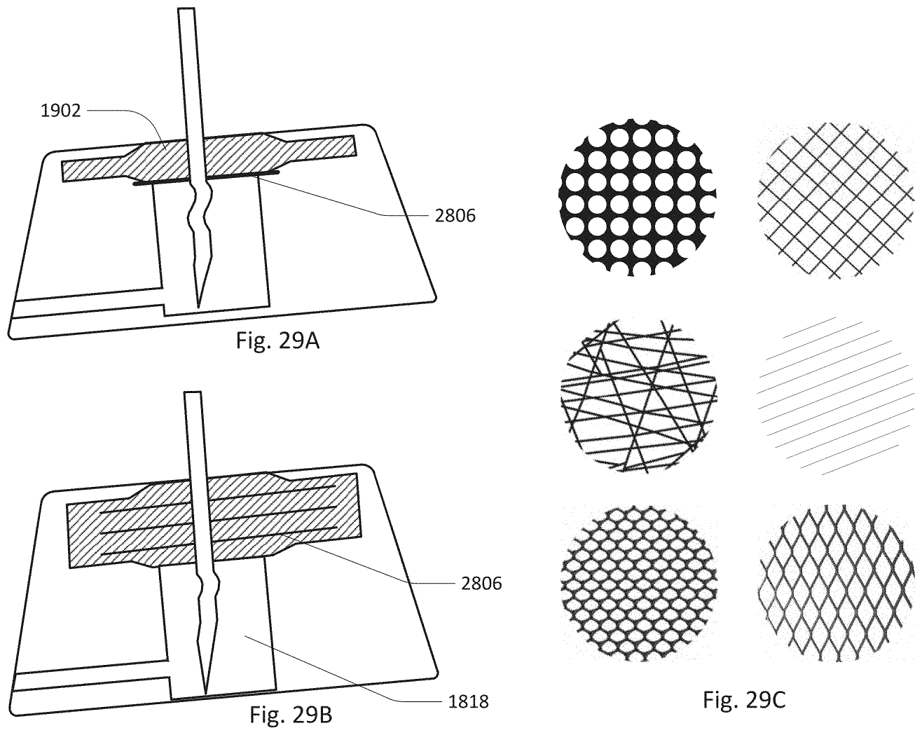

FIGS. 30A-F show alternative needle capture embodiments of the insulin delivery system. FIG. 30A includes a needle with threads 3002 incorporated into the wall of the needle. This embodiment can be advanced through the skin and through the septum using a rotational force, like a screw. Once the needle is in place in the reservoir, and the patch is applied to the skin, the needle is unlikely to be able to slide back and forth across the septum because of the threads. The patch prevents any rotation of the needle with respect to the subcutaneous port/septum.

FIG. 30B shows an embodiment similar to that in FIG. 30A except that the threads are added to the outside of the needle. In some embodiments, a hydrophilic, or other polymer may be placed between the coils so that the outer surface of the needle is smooth. In these embodiments, the polymer may swell over time, or once it is in place in the reservoir. This swelling will create a non-smooth surface which will prevent the needle from coming out accidentally, but will still allow the needle to be removed with some small force.

FIGS. 30C and 30D show an embodiment which includes cannula 3006, similar to the cannula shown in FIGS. 23 and 24. The cannula is left behind after the needle is removed. In this embodiment, the cannula is made out of swelling material, such as a hydrophilic polymer, and its OD swells slightly over time after it has been placed into the reservoir. The ID of the swelling cannula remains large enough to infuse insulin. The entire length of the cannula may be made of a swelling material, or only a portion. A swelling material may be added to a needle as an alternative embodiment.

FIG. 30E shows an embodiment which includes a cannula, or a needle, which includes balloon or expansion member 3008. In this embodiment, the balloon is inflated after the needle/cannula is in place in the reservoir. The larger OD of the balloon prevents the needle from slipping out of the port. One or more balloons may be present. The balloon may be inflated/deflated via a lumen in the cannula/needle. The balloon may be deflated before the cannula/needle is removed. Multiple balloons may utilize a single inflation lumen, or have multiple inflation lumens.

FIG. 30F shows an embodiment similar to that shown in FIG. 30E, except that in this embodiment the balloon/expandable member is larger. In this embodiment the balloon, when inflated, fills up the majority of the reservoir. This reduces the volume of insulin in the system and allows for easier flushing and/or priming of the system. The balloons in the various embodiments may be made from a compliant or non-compliant material and may be incorporated into a needle or cannula. In the embodiment shown in FIG. 30F, the balloon is preferably made from a relatively non-compliant material so that the volume of the balloon can be better controlled.

In some embodiments, a balloon/expandable member may be used to flush the system of insulin. For example, a balloon similar to that shown in FIG. 30F may be inflated to displace virtually all of the volume of the reservoir, thus purging the reservoir of its contents. It may be deflated, completely, or only partially for insulin delivery.

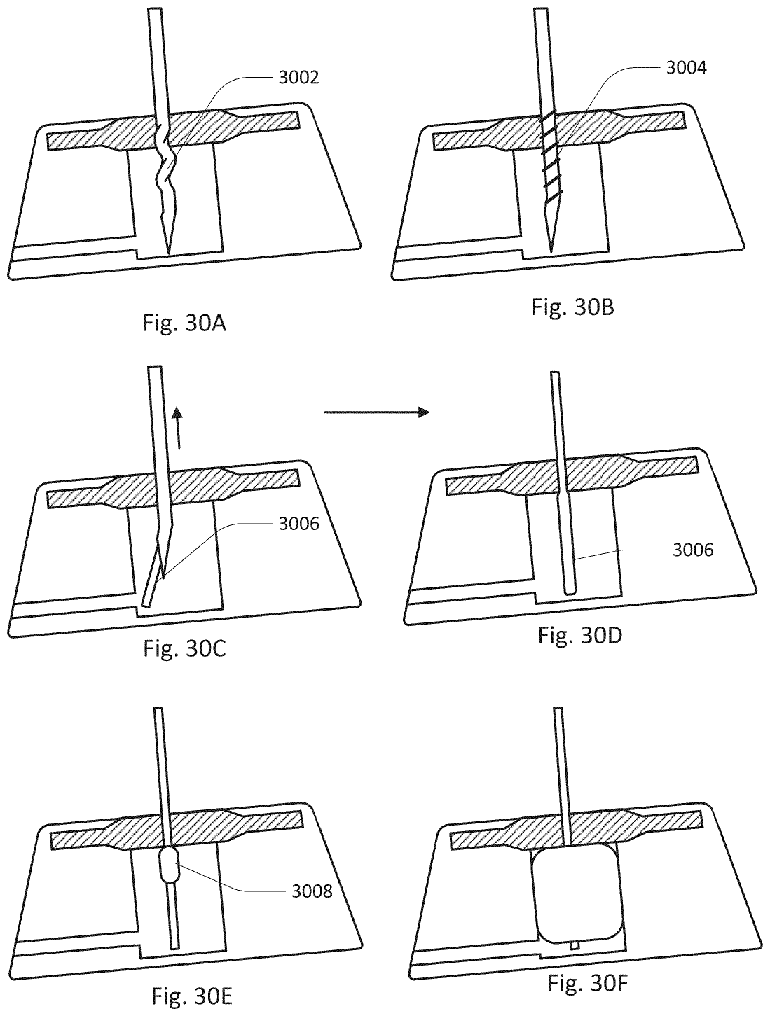

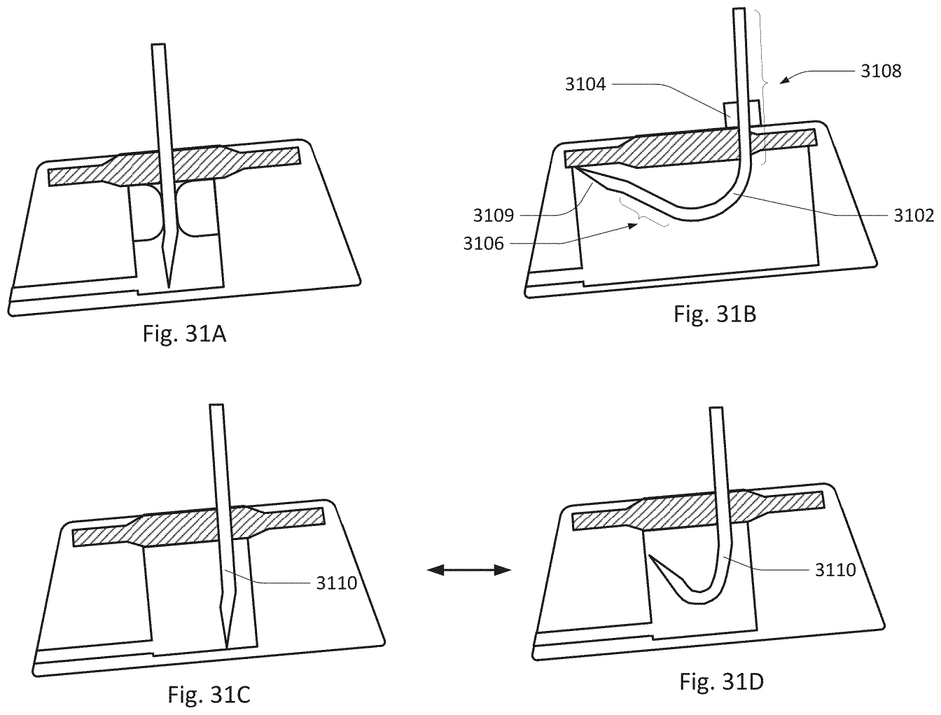

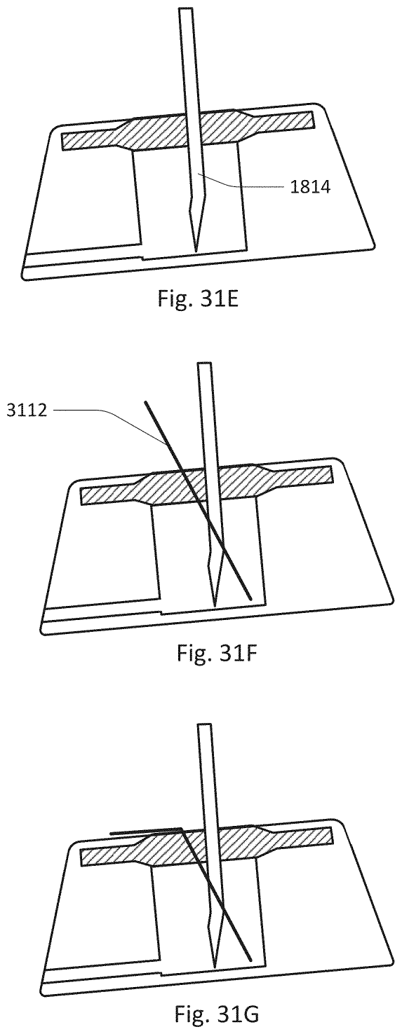

FIG. 31A shows an embodiment where a balloon/expandable member is incorporated into the subcutaneous port reservoir instead of the cannula/needle.

FIG. 31B shows an embodiment which incorporates curved needle 3102. The curved needle is placed through the skin and into the subcutaneous port by holding the tip of the needle generally perpendicularly to the skin, and inserting the needle, angling as it is being inserted so that the needle is generally perpendicular to the skin during insertion. This involves angling the needle with respect to the surface of the skin as the needle is inserted. Once the needle is inserted, the curve of the tip of the needle helps prevent the needle from accidentally coming out of the subcutaneous port, as the needle will need to follow the same angles to be removed. To prevent the needle from accidentally moving to an angle allowing removal, stabilizer 3104 may be placed on the needle. The stabilizer may be incorporated into the adhesive patch component which holds the needle in place, or the stabilizer may be a separate component. The stabilizer prevents the needle from moving to an angle which will allow its removal. When the needle is to be removed on purpose, the stabilizer/patch may be removed. FIG. 31B shows an embodiment where curved needle 3102 includes at least two relatively straight portions, 3106 and 3108, although a curved needle may have one, or more than two straight portions. Also in this figure opening 3109 of the needle is facing away from the septum when the curved needle is in place, although it may face upward, or sideways.

Different curve shapes and/or lengths may be used for people with different amounts of fat, or for different locations on the body. Also, different curve shapes and/or lengths may be used for subsequent punctures with one subcutaneous port to avoid puncturing in the same area of the septum.

FIGS. 31C and 31D show an embodiment that includes shape memory needle 3110, such as a nitinol needle. The needle may be straight when it is at room temperature and take on a different shape, such as a curve, as shown here, or other shape, when the needle is exposed to body temperature. To remove the needle, either extra force or angling may be required, or alternatively, cold saline may be used to flush the system and straighten the needle.

FIGS. 31E-G show an embodiment utilizing a locking needle. Needle 1814 is pierced through the septum, and then locking needle 3112 is placed through the septum at a different angle to contact and engage needle 1814, locking it in place in the reservoir. Locking needle 3112 is preferably small, and solid, not hollow, and may be bent so that it may be flattened, as shown in FIG. 31G. To remove needle 1814, locking needle 3112 is removed first, then needle 1814 is removed.

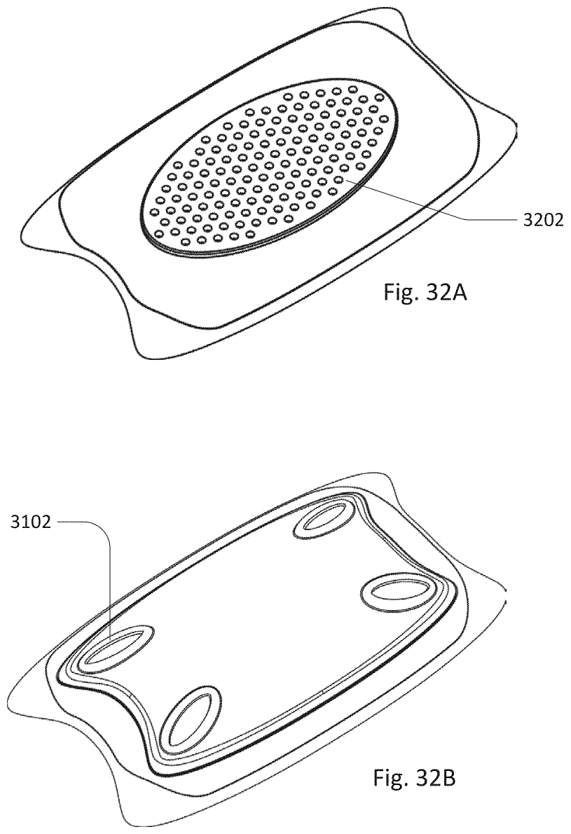

FIGS. 32A and B show an embodiment of a piercable septum which includes mesh 3202 on the bottom (reservoir facing) side of the septum, and palpable points 3204 on the top (skin facing) side of the septum. The septum may be made out of silicone or other suitable material, and the mesh may be made out of metal, polymer or other suitable material. Points 3204 may be felt through the skin when the subcutaneous port is implanted.

Embodiments disclosed herein which include an access device or needle for percutaneous access to a subcutaneous port may be designed to be worn continuously, for example for several days, before the access device/needle needs to be replaced. For example, the access device/needle (which may be incorporated into a patch) may be designed to be in place for up to 7 days. For example, the access device/needle may be designed to be in place for 1-3 days. For example, the access device/needle may be designed to be in place for 1-7 days. For example, the access device/needle may be designed to be in place for up to 10 days. For example, the access device/needle may be designed to be in place for up to 20 days. For example, the access device/needle may be designed to be in place for more than 1 hour. For example, the access device/needle may be designed to be in place for more than 1 day. For example, the access device/needle may be designed to be in place for more than 2 days. For example, the access device/needle may be designed to be in place for more than 3 days. For example, the access device/needle may be designed to be in place for more than 4 days. For example, the access device/needle may be designed to be in place for more than 5 days. For example, the access device/needle may be designed to be in place for more than 7 days.

Embodiments disclosed herein which include external insulin pumps may alternatively incorporate an implantable insulin pump. An insulin designed for peritoneal delivery may be used in the insulin pump. Insulin pumps may include a basal insulin delivery rate as well as the ability to deliver bolus amounts of insulin. The boluses may be delivered manually, or automatically, and the bolus size may be based on measured glucose levels, or estimates based on food/carbohydrates consumed.

Basal infusion rates (ongoing, automatic) may range from 0.001 to 15 units/hour, where there are 100 units of insulin per milliliter (ml) of liquid, or 500 units of insulin per milliliter (ml) of liquid. Alternatively the basal infusion rates may range from 0.01 to 30 units/hour. Bolus infusion rates may range from 0.01 to 50 units.

Lumen patency of the insulin delivery systems may be tested by a pressure sensor in the insulin pump, or elsewhere in the system. A test injection of saline or other inert fluid may be used to test the fluid path between the insulin pump and the peritoneal cavity. The pressure within the lumen may be measured to determine whether a blockage is present. A saline injection may also be used following an insulin injection to force any stagnant insulin out of the reservoir of the subcutaneous port and into the peritoneal cavity. A one way valve may also be present within the infusion tubing, patch, needle, subcutaneous port or insulin delivery tubing to prevent backflow of fluids. Alternatively the valve may be mechanical, and triggered by a switch or other mechanism. The embodiments disclosed herein may be used for insulin delivery and glucose sensing. They may also be used for any type of drug or fluid delivery, and/or any type of analyte sensing, including sodium, potassium, chloride, bicarbonate, urea, creatinine, triglyceride, protein, albumin, hemoglobin, oxygen, ketones, LDL, HDL, cholesterol, etc.

In some embodiments, reservoir 1818 may have a dynamic volume. In other words, it may be designed to expand and/or contract. For example, it may be expanded for introduction of the needle into the reservoir and then contract after the needle is in place to decrease the volume of the reservoir.

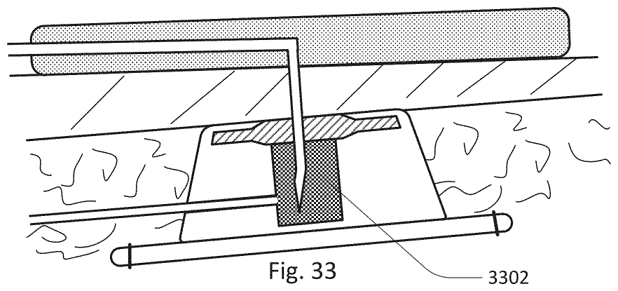

In some embodiments, a foam, or lattice may be incorporated into the insulin reservoir, as shown in FIG. 33. Foam 3302 takes up the majority of the volume in the insulin reservoir, which allowing a large area for the needle. This reduces the volume of insulin in the entire system which facilitates flushing of the system. The foam would allow insulin to be injected through the needle and into the reservoir and through the catheter and into the peritoneal cavity.

What is claimed is:

1. A port apparatus configured for placement within a body of a subject, comprising:
a port housing;
a catheter defining a first lumen which is fluidly coupled to the port housing;
a flushing lumen extending from the port housing and terminating at or near a distal tip of the catheter;
an access port positioned within or upon the port housing and in fluid communication with the first lumen; and
an access device configured for percutaneous advancement into contact with the access port.

2. The apparatus of claim 1 wherein the port apparatus is configured for subcutaneous placement within the body.

3. The apparatus of claim 1 further comprising a reservoir configured to be placed into fluid communication within the access port.

4. The apparatus of claim 3 wherein the reservoir comprises a syringe.

5. The apparatus of claim 3 wherein the reservoir contains insulin.

6. The apparatus of claim 1 wherein the catheter comprises a distal tip which is configured for positioning within a peritoneal space of the body.

7. The apparatus of claim 6 wherein the distal tip is configured for positioning within a true pelvis of the peritoneal space.

8. The apparatus of claim 1 further comprising an analyte sensor positioned to be in communication with a fluid within the body.

9. The apparatus of claim 8 wherein the analyte sensor is positioned within the port housing.

10. The apparatus of claim 8 wherein the analyte sensor is positioned within or upon the catheter.

11. The apparatus of claim 1 further comprising a controller in communication with the port apparatus.

12. The apparatus of claim 11 wherein further comprising an analyte sensor positioned within the controller.

13. The apparatus of claim 11 wherein the controller is configured to control an infusion of insulin through the catheter and into the body and/or monitor a glucose level of the body.

14. The apparatus of claim 11 wherein the controller is configured to infuse a fluid through the catheter in a closed loop or semi-closed loop based upon a parameter sensed within a fluid contained within the body.

15. The apparatus of claim 14 wherein the controller is configured to monitor a parameter of a peritoneal fluid drawn from within a peritoneal cavity of the body.

16. The apparatus of claim 11 further comprising an analyte sensor in communication with the controller for detecting at least one parameter within a fluid received through the lumen.

17. The apparatus of claim 11 wherein the controller is configured for maintaining the access device in place within the access port for at least 1 hour.

18. The apparatus of claim 11 wherein the controller is configured for maintaining the access device in place within the access port continuously.

19. The apparatus of claim 11 wherein the controller is configured to periodically provide a supply of insulin and to continuously detect a glucose level of the body via the access device.

20. The apparatus of claim 16 wherein the analyte sensor comprises a glucose sensor.

21. The apparatus of claim 16 wherein the analyte sensor is incorporated into the catheter.

22. The apparatus of claim 16 wherein the analyte sensor is incorporated into the port housing.

23. The apparatus of claim 16 wherein the analyte sensor is positioned remotely from the port apparatus.

24. The apparatus of claim 1 wherein the access device comprises at least one conductive portion configured to be in electrical communication with the access port.

25. The apparatus of claim 24 wherein the access device comprises two or more conductive portions.

26. The apparatus of claim 25 wherein the two or more conductive portions are configured to be in electrical communication with two or more conductive portions of the access port in a corresponding manner.

27. The apparatus of claim 1 wherein the access device further comprises a patch configured for securement to a skin surface of the subject.

28. The apparatus of claim 1 wherein the access device comprises a needle or cannula having at least one conductive portion configured to be in electrical communication with the access port.

29. The apparatus of claim 1 further comprising a flushing mechanism in fluid communication with the catheter, wherein the flushing mechanism is configured to apply a negative and/or positive pressure via the catheter.

30. A port apparatus, comprising:
a port housing configured for subcutaneous placement within a body of a subject;
a catheter defining a lumen which is fluidly coupled to the port housing;
a flushing lumen extending from the port housing and terminating at or near a distal tip of the catheter;
an access port positioned within or upon the port housing and in fluid communication with the lumen;
an analyte sensor in electrical communication with the access port;
an access device configured for percutaneous advancement into contact with the access port such that the access device and access port are in electrical communication when the access device is received within the access port; and
a controller in electrical communication with the access device such that the controller is in communication with the analyte sensor when the access device is in electrical communication with the access port.

31. The apparatus of claim 30 wherein the access device comprises at least one conductive portion configured to be in electrical communication with the access port.

32. The apparatus of claim 31 wherein the access port comprises at least one conductive portion configured to contact the at least conductive portion of the access device.

33. The apparatus of claim 30 wherein the access device comprises two or more conductive portions.

34. The apparatus of claim 33 wherein the two or more conductive portions are configured to be in electrical communication with two or more conductive portions of the access port in a corresponding manner.

35. The apparatus of claim 30 further comprising a reservoir configured to be placed into fluid communication within the access port.

36. The apparatus of claim 35 wherein the reservoir comprises a syringe.

37. The apparatus of claim 35 wherein the reservoir contains insulin.

38. The apparatus of claim 30 wherein the catheter comprises a distal tip which is configured for positioning within a peritoneal space of the body.

39. The apparatus of claim 38 wherein the distal tip is configured for positioning within a true pelvis of the peritoneal space.

40. The apparatus of claim 30 further comprising a flushing mechanism in fluid communication with the catheter, wherein the flushing mechanism is configured to apply a negative and/or positive pressure via the catheter.

41. The apparatus of claim 30 wherein the analyte sensor comprises a glucose sensor.

42. The apparatus of claim 30 wherein the analyte sensor is incorporated into the catheter.

43. The apparatus of claim 30 wherein the analyte sensor is incorporated into the port housing.

44. The apparatus of claim 30 wherein the controller is configured to control an infusion of insulin through the catheter and into the body and/or monitor a glucose level of the body.

45. The apparatus of claim 30 wherein the controller is configured to infuse a fluid through the catheter in a closed loop or semi-closed loop based upon a parameter sensed within a fluid contained within the body.

46. The apparatus of claim 30 wherein the controller is configured to monitor a parameter of a peritoneal fluid drawn from within a peritoneal cavity of the body.

47. The apparatus of claim 30 further comprising an analyte sensor in communication with the controller for detecting at least one parameter within a fluid in contact with the analyte sensor.

48. The apparatus of claim 30 wherein the access port comprises a plurality of conductive mesh sheets.

49. The apparatus of claim 48 wherein the plurality of conductive mesh sheets are configured to be aligned in a corresponding manner with the access device.

50. A method of controlling an infusion of fluid within a body of a subject, comprising:
receiving an access device advanced percutaneously into contact with an access port positioned within or upon a port housing, wherein the port housing is positioned subcutaneously within a body of a subject;
forming an electrical contact between at least one conductive portion of the access device and at least one conductive portion of the access port;
contacting a fluid from within the body with an analyte sensor;
receiving a signal from the analyte sensor in contact with the fluid; and
determining at least one parameter from the fluid relating to the body via a controller in electrical communication with the analyte sensor.

51. The method of claim 50 wherein the access device comprises at least one conductive portion configured to be in electrical communication with the access port.

52. The method of claim 50 further comprising drawing the fluid from within a peritoneal cavity into a catheter fluidly coupled to the access port.

53. The method of claim 50 wherein the access port comprises at least one conductive portion configured to contact the at least conductive portion of the access device.

54. The method of claim 50 wherein forming an electrical contact comprises electrically contacting two or more conductive portions along the access device with two or more conductive portions of the access port in a corresponding manner.

55. The method of claim 50 further comprising introducing a volume of insulin through the catheter and into the body.

56. The method of claim 55 wherein the volume of insulin is based upon the at least one parameter.

57. The method of claim 50 wherein the catheter comprises a distal tip which is configured for positioning within a peritoneal space of the body.

58. The method of claim 57 wherein the distal tip is configured for positioning within a true pelvis of the peritoneal space.

59. The method of claim 50 further comprising flushing a second fluid into the catheter.

60. The method of claim 50 wherein determining at least one parameter comprises determining a glucose level within the body.
